SEQUENCE LISTING (57 sequences)

SEQ ID NO: 1 - protein, 256 aa, synthetic construct
QVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGEGGGGEGGGGEVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 2 - protein, 10 aa, synthetic construct
GRISSIIHMA

SEQ ID NO: 3 - protein, 13 aa, synthetic construct
RVGTTVYADSVKG

SEQ ID NO: 4 - protein, 11 aa, synthetic construct
LQYEKHGGADY

SEQ ID NO: 5 - protein, 8 aa, synthetic construct
GRPVSNYA

SEQ ID NO: 6 - protein, 8 aa, synthetic construct
INWQKTAT

SEQ ID NO: 7 - protein, 17 aa, synthetic construct
AAVFRVVAPKTQYDYDY

SEQ ID NO: 8-9 - skipped (2 entries)

SEQ ID NO: 10 - protein, 15 aa, synthetic construct
GGGGEGGGGEGGGGE

SEQ ID NO: 11 - protein, 15 aa, synthetic construct
GGGGSGGGGSGGGGS

SEQ ID NO: 12 - protein, 5 aa, synthetic construct
GGGGS

SEQ ID NO: 13 - protein, 10 aa, synthetic construct
GGGGSGGGGS

SEQ ID NO: 14 - protein, 15 aa, synthetic construct
GGGGSGGGGSGGGGS

SEQ ID NO: 15 - protein, 20 aa, synthetic construct
GGGGSGGGGSGGGGSGGGGS

SEQ ID NO: 16 - protein, 25 aa, synthetic construct
GGGGSGGGGSGGGGSGGGGSGGGGS

SEQ ID NO: 17 - protein, 30 aa, synthetic construct
GGGGSGGGGSGGGGSGGGGSGGGGSGGGGS

SEQ ID NO: 18 - protein, 15 aa, synthetic construct
EAAAKEAAAKEAAAK

SEQ ID NO: 19 - protein, 5 aa, synthetic construct
PAPAP

SEQ ID NO: 20 - protein, 10 aa, synthetic construct
GGGGSPAPAP

SEQ ID NO: 21 - protein, 10 aa, synthetic construct
PAPAPGGGGS

SEQ ID NO: 22 - protein, 10 aa, synthetic construct
GGGDSGGGDS

SEQ ID NO: 23 - protein, 10 aa, synthetic construct
GGGESGGGES

SEQ ID NO: 24 - protein, 10 aa, synthetic construct
GGGDSGGGGS

SEQ ID NO: 25 - protein, 10 aa, synthetic construct
GGGASGGGGS

SEQ ID NO: 26 - protein, 10 aa, synthetic construct
GGGESGGGGS

SEQ ID NO: 27 - protein, 6 aa, synthetic construct
ASTKGP

SEQ ID NO: 28 - protein, 13 aa, synthetic construct
ASTKGPSVFPLAP

SEQ ID NO: 29 - protein, 4 aa, synthetic construct
GGGP

SEQ ID NO: 30 - protein, 8 aa, synthetic construct
GGGGGGGP

SEQ ID NO: 31 - protein, 9 aa, synthetic construct
PAPNLLGGP

SEQ ID NO: 32 - protein, 6 aa, synthetic construct
GGGGGG

SEQ ID NO: 33 - protein, 12 aa, synthetic construct
GGGGGGGGGGGG

SEQ ID NO: 34 - protein, 8 aa, synthetic construct
APELPGGP

SEQ ID NO: 35 - protein, 8 aa, synthetic construct
SEPQPQPG

SEQ ID NO: 36 - protein, 15 aa, synthetic construct
GGGSSGGGSSGGGSS

SEQ ID NO: 37 - protein, 14 aa, synthetic construct
GGGGGGGGGSGGGS

SEQ ID NO: 38 - protein, 15 aa, synthetic construct
GGGGSGGGGGGGGGS

SEQ ID NO: 39 - protein, 15 aa, synthetic construct
GGSSSGGSSSGGSSS

SEQ ID NO: 40 - protein, 15 aa, synthetic construct
GSSSSGSSSSGSSSS

SEQ ID NO: 41 - protein, 15 aa, synthetic construct
GGGGAGGGGSGGGGS

SEQ ID NO: 42 - protein, 15 aa, synthetic construct
GGGGSGGGGAGGGGS

SEQ ID NO: 43 - protein, 15 aa, synthetic construct
GGGASGGGGSGGGGS

SEQ ID NO: 44 - protein, 14 aa, synthetic construct
GGGGSGGGAGGGGS

SEQ ID NO: 45 - protein, 15 aa, synthetic construct
GGGGSAGGGSGGGGS

SEQ ID NO: 46 - protein, 15 aa, synthetic construct
GGGGSGGGGSAGGGS

SEQ ID NO: 47 - protein, 15 aa, synthetic construct
GGGGSAGGGSAGGGS

SEQ ID NO: 48 - protein, 15 aa, synthetic construct
GGGGDGGGGSGGGGS

SEQ ID NO: 49 - protein, 15 aa, synthetic construct
GGGGSGGGGDGGGGS

SEQ ID NO: 50 - protein, 15 aa, synthetic construct
GGGGDGGGGDGGGGS

SEQ ID NO: 51 - protein, 15 aa, synthetic construct
GGGGEGGGGSGGGGS

SEQ ID NO: 52 - protein, 15 aa, synthetic construct
GGGGSGGGGEGGGGS

SEQ ID NO: 53 - protein, 15 aa, synthetic construct
GGGGEGGGGEGGGGS

SEQ ID NO: 54 - protein, 15 aa, synthetic construct
GGGGAGGGGAGGGGS

SEQ ID NO: 55 - protein, 123 aa, synthetic construct
QVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 56 - protein, 118 aa, synthetic construct
VQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 57 - protein, 774 aa, synthetic construct
CAGGTGCAGCTGGTGGAAAGCGGCGGAGGCCTGGTCAAGCCTGGCGGCAGCCTGAGACTGAGCTGTGCCGCCAGCGGCAGACCCGTGTCCAATTACGCCGCTGCCTGGTTCCGGCAGGCCCCTGGCAAAGAGAGAGAGTTCGTCAGCGCCATCAACTGGCAGAAAACCGCCACCTACGCCGACAGCGTGAAGGGCCGGTTCACCATCAGCCGGGACAACGCCAAGAACAGCCTGTACCTGCAGATGAACTCCCTGCGGGCCGAGGACACCGCCGTGTACTACTGCGCCGCTGTGTTCCGGGTGGTGGCCCCCAAGACCCAGTACGACTACGATTACTGGGGCCAGGGCACCCTGGTCACCGTGTCATCTGGCGGAGGGGGAGAAGGCGGGGGAGGGGAAGGGGGAGGCGGCGAAGTCCAGCTGCTGGAATCTGGGGGCGGACTGGTGCAGCCAGGCGGCTCCCTCAGACTGTCTTGCGCCGCCTCCGGCCGGATCAGCAGCATCATCCACATGGCCTGGTTTAGACAGGCTCCCGGAAAAGAACGCGAGCTGGTGTCCGAGATCTCCAGAGTGGGCACCACCGTGTATGCCGACTCCGTGAAAGGCAGATTCACAATCTCCCGCGACAACAGCAAGAATACTCTGTATCTCCAGATGAATAGCCTGAAGCCCGAAGATACAGCCGTCTACTATTGCAACGCCCTGCAGTACGAGAAGCACGGCGGAGCCGACTATTGGGGACAGGGAACACTCGTGACAGTGTCTAGCTGATGA

(19) United States
(12) Patent Application Publication
(10) Pub. No.: US 2026/0317409 A1
(43) Pub. Date: Oct. 15, 2026

(54) PHARMACEUTICAL COMPOSITIONS OF FUSION PROTEINS AND METHODS OF USE THEREOF

(71) Applicant: Alexion Pharmaceuticals, Inc., Boston, MA (US)

(72) Inventors: Teng-Chieh YANG, Boston, MA (US); Huan KANG, Boston, MA (US); Vinay RADHAKRISHNAN, Boston, MA (US)

(21) Appl. No.: 19/101,320

(22) PCT Filed: Aug. 4, 2023
(86) PCT No.: PCT/US2023/071667

§ 371 Date: Feb. 5, 2025

Related U.S. Application Data
(60) Provisional application No. 63/395,417, filed on Aug. 5, 2022.

(51) Int. Cl. C07K 16/18; A61K 47/02; A61K 47/12; A61K 47/18; A61K 47/26

(52) U.S. Cl. CPC C07K 16/18; A61K 47/02; A61K 47/12; A61K 47/183; A61K 47/26; C07K 2317/31

(57) ABSTRACT

The present disclosure provides pharmaceutical compositions formulated for high concentrations of fusion proteins for subcutaneous administration and methods of use thereof. Furthermore, the disclosure provides methods of developing high concentration protein formulations.

### SEQUENCE LISTING

The instant application contains a Sequence Listing which has been submitted electronically in XML format and is hereby incorporated by reference in its entirety. Said XML copy, created on Aug. 3, 2023, is named 51196-031 WO2_Sequence_Listing_8_3_23.xml and is 50,310 bytes in size.

### BACKGROUND

Subcutaneous administration of protein therapeutics offers several advantages over other methods of administration. For example, subcutaneous administration has been shown to improve patient compliance, improve convenience, and improve the pharmacokinetic and pharmacodynamic profile. However, one of the challenges with developing formulations for subcutaneous administration is that the injection volume is typically less than 2 mL. As a result, the protein in the pharmaceutical composition must be present in a high concentration. Accordingly, there remains a need for high concentration formulations to achieve the appropriate dose. Several challenges remain associated with developing a high concentration formulation such as viscosity, stability and delivery.

### SUMMARY

In a first aspect, the disclosure provides a pharmaceutical composition including a fusion protein, detergent, and a buffering agent, wherein the fusion protein is at a concentration of at least 120 mg/mL. In some embodiments, the fusion protein includes the complementarity-determining regions (CDRs) having amino acid sequences of SEQ ID NOS: 2-7 In some embodiments, the fusion protein includes a first portion including an amino acid sequence having at least 95% (e.g., at least 96%, 97%, 98%, 99%, or 100%) sequence identity to SEQ ID NO: 55 and a second portion including an amino acid sequence having at least 95% (e.g., at least 96%, 97%, 98%, 99%, or 100%) sequence identity SEQ ID NO: 56. In some embodiments, the fusion protein consists of the amino acid sequence of SEQ ID NO: 1 or a modification thereof. In some embodiments, the modification comprises conversion of the N-terminal glutamine of the sequence of SEQ ID NO: 1 to pyro-glutamate.

In some embodiments, the concentration of the fusion protein is between 120 mg/mL and 250 mg/mL (e.g., between 120 mg/mL and 250 mg/mL, 150 mg/mL and 250 mg/mL, 175 mg/mL and 250 mg/mL, 200 mg/mL and 250 mg/mL, 225 mg/mL and 250 mg/mL, 120 mg/mL and 225 mg/mL, 120 mg/mL and 200 mg/mL, 120 mg/mL and 175 mg/mL, 120 mg/mL and 150 mg/mL, or 150 mg/mL and 180 mg/mL). In some embodiments, the fusion protein is at a concentration of at least 150 mg/mL. In some embodiments, the concentration of the fusion protein is between 150 mg/mL and 200 mg/mL (e.g., between 150 mg/mL and 200 mg/mL, 150 mg/mL and 190 mg/mL, 150 mg/mL and 180 mg/mL, 150 mg/mL and 170 mg/mL, 150 mg/mL and 160 mg/mL, 160 mg/mL and 200 mg/mL, 170 mg/mL and 200 mg/mL, 180 mg/mL and 200 mg/mL, or 190 mg/mL and 200 mg/mL) or higher, e.g., 250 mg/mL. In some embodiments, the concentration of the fusion protein is between 170 mg/mL and 200 mg/mL (e.g., between 170 mg/mL and 195 mg/mL, 170 mg/mL and 190 mg/mL, 170 mg/mL and 185 mg/mL, 170 mg/mL and 180 mg/mL, 170 mg/mL and 175 mg/mL, 175 mg/mL and 200 mg/mL, 180 mg/mL and 200 mg/mL, 185 mg/mL and 200 mg/mL, 190 mg/mL and 200 mg/mL, or 195 mg/mL and 200 mg/mL). In some embodiments, the concentration of the fusion protein is about 190 mg/mL. In some embodiments, the concentration of the fusion protein is about 150 mg/mL.

In some embodiments, the detergent is a polysorbate. In some embodiments, the polysorbate is polysorbate 80 (PS80). In some embodiments, the concentration of the polysorbate is between 0.001% and 1% (w/v) (e.g., between 0.001% (w/v) and 0.8% (w/v), 0.001% (w/v) and 0.6% (w/v), 0.001% (w/v) and 0.4% (w/v), 0.001% (w/v) and 0.2% (w/v), 0.001% (w/v) and 0.05% (w/v), 0.005% (w/v) and 0.1% (w/v), 0.1% (w/v) and 1.5% (w/v), 0.3% (w/v) and 1.5% (w/v), 0.4% (w/v) and 1.5% (w/v), 0.005% (w/v) and 1% (w/v), 0.05% (w/v) and 1% (w/v), or 0.1% (w/v) and 1% (w/v)). In some embodiments, the concentration of polysorbate is about 0.05% (w/v). In some embodiments, the concentration of the polysorbate is between 0.05% (w/v) and 0.5% (w/v) (e.g., between 0.05% (w/v) and 0.3% (w/v), 0.05% (w/v) and 0.1% (w/v), 0.1% (w/v) and 0.5% (w/v), 0.2% (w/v) and 0.5% (w/v), or 0.3% (w/v) and 0.5% (w/v)). In some embodiments, the concentration of the polysorbate is between 0.1% (w/v) and 0.2% (w/v) (e.g., between 0.1% (w/v), 0.11% (w/v), 0.12% (w/v), 0.13% (w/v), 0.14% (w/v), 0.15% (w/v), 0.16% (w/v), 0.17% (w/v), 0.18% (w/v), 0.19% (w/v), or 0.2% (w/v). In some embodiments the concentration of the polysorbate is about 0.15% (w/v).

In some embodiments, the detergent is at a concentration between 0.01% (w/v) and 1.5% (w/v) (e.g., between 0.01% (w/v) and 0.4% (w/v), 0.01% (w/v) and 0.3% (w/v), 0.01% (w/v) and 0.2% (w/v), 0.01% (w/v) and 0.1% (w/v), 0.01% (w/v) and 1% (w/v), 0.1% (w/v) and 1.5% (w/v), 0.2% (w/v) and 1.5% (w/v), 0.3% (w/v) and 1.5% (w/v), 0.4% (w/v) and 1.5% (w/v), 0.5% (w/v) and 1.5% (w/v), 0.8% (w/v) and 1.5% (w/v), or 1% (w/v) and 1.5% (w/v)). In some embodiments, the detergent is at a concentration between 0.01% (w/v) and 1% (w/v) (e.g., 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), or 1% (w/v)). In some embodiments, the detergent is at a concentration of between about 0.05% (w/v) and 0.1% (w/v) (e.g., 0.05% (w/v), 0.06% (w/v), 0.07% (w/v), 0.08% (w/v), 0.09% (w/v), or 0.1% (w/v)). In some embodiments, the detergent is at a concentration of about 0.05% (w/v). In some embodiments, the detergent is at a concentration of about 0.1% (w/v).

In some embodiments, the buffering agent is an acetate, a histidine, a phosphate, or a succinate, or a combination thereof. In some embodiments, the buffering agent is an acetate. In some embodiments, the acetate is sodium acetate. In some embodiments, the concentration of the sodium acetate is between about 10 mM and 150 mM (e.g., between 10 mM and 140 mM, 10 mM and 130 mM, 10 mM and 120 mM, 10 mM and 100 mM, 10 mM and 75 mM, 10 mM and 50 mM, 20 mM and 150 mM, 30 mM and 150 mM, 75 mM and 150 mM, 100 mM and 150 mM or 40 mM and 150 mM). In some embodiments, the concentration of the sodium acetate is between about 15 mM and 100 mM (e.g., between about 15 mM and 80 mM, 15 mM and 60 mM, 15 mM and 40 mM, 15 mM and 20 mM, 20 mM and 100 mM, 40 mM and 100 mM, 60 mM and 100 mM, or 80 mM and 100 mM. In some embodiments, the concentration of the sodium acetate is about 50 mM. In some embodiments, the concentration of the sodium acetate is about 20 mM.

In some embodiments, the pharmaceutical composition further includes an amino acid. In some embodiments, the amino acid is proline. In some embodiments, the amino acid is arginine. In some embodiments, the amino acid is glycine. In some embodiments, the amino acid is present in the pharmaceutical composition at concentration between 100 mM and 200 mM (e.g., between 100 mM and 190 mM, 100 mM and 180 mM, 100 mM and 170 mM, 140 mM and 200 mM, 150 mM and 200 mM, or 160 mM and 200 mM, 110 mM and 190 mM, 120 mM and 180 mM, 130 mM and 170 mM, 140 mM and 180 mM, 150 mM and 170 mM or 160 mM and 170 mM). In particular embodiments, the concentration of the amino acid is about 165 mM.

In some embodiments, the amino acid has a concentration of between about 10 mM and 200 mM (e.g., between 10 mM and 150 mM, 10 mM and 100 mM, 10 mM and 50 mm, 10 mM and 20 mM, 20 mM and 200 mM, 50 mM and 200 mM, 100 mM and 200 mM, or 150 mM and 200 mM). In some embodiments, the amino acid has a concentration of between about 100 mM and 200 mM (e.g., between 100 mM and 180 mM, 100 mM and 160 mM, 100 mM and 140 mM, 100 mM and 120 mM, 120 mM and 200 mM, 140 mM and 200 mM, 160 mM and 200 mM, or 180 mM and 200 mM.

In some embodiments, the pharmaceutical composition further includes a tonicity agent. In some embodiments, the tonicity agent is a sugar, an amino acid, or a salt. In some embodiments, the salt is NaCl. In some embodiments, the sugar is sucrose, glucose, glycerol, or trehalose. In some embodiments, the sugar is sucrose. In some embodiments, the sucrose is at a concentration between about 1% (w/v) and about 15% (w/v) (e.g., 1% (w/v), 2% (w/v), 3% (w/v), 4% (w/v), 5% (w/v), 6% (w/v), 7% (w/v), 8% (w/v), 9% (w/v), 10% (w/v), 11% (w/v), 12% (w/v), 13% (w/v), 14% (w/v), or 15% (w/v)). In some embodiments, the sucrose is present at a concentration between 2% (w/v) and 10% (w/v) (e.g., 2% (w/v), 3% (w/v), 4% (w/v), 5% (w/v), 6% (w/v), 7% (w/v), 8% (w/v), 9% (w/v), or 10% (w/v)). In some embodiments, the sucrose is at a concentration between about 4% (w/v) and about 9% (w/v) (e.g., 4% (w/v), 5% (w/v), 6% (w/v), 7% (w/v), 8% (w/v), 9% (w/v), or 10% (w/v)). In some embodiments, the sucrose is at a concentration of about 4% (w/v). In some embodiments, the sucrose is present at a concentration between 8% (w/v) and 9% (w/v) (e.g., 8.1% (w/v), 8.2% (w/v), 8.3% (w/v), 8.4% (w/v), 8.5% (w/v), 8.6% (w/v), 8.7% (w/v), 8.8% (w/v), 8.9% (w/v), or 9% (w/v)). In some embodiments, the sucrose is present at a concentration of about 8.6% (w/v).

In some embodiments, the pharmaceutical composition has a pH of between about pH 3 and pH 8 (e.g., between pH 3 and pH 6, pH 3 and pH 4, pH 4 and pH 8, or pH 6 and pH 8). In some embodiments, the pharmaceutical composition has a pH of between pH 4 and pH 7 (e.g., between pH 4 and pH 6, pH 4 and pH 5, pH 5 and pH 7, or pH 6 and pH 7). In some embodiments, the pharmaceutical composition has a pH of about pH 5.4.

In some embodiments, the pharmaceutical composition has a viscosity of <17 cP at 20° C. In some embodiments, the pharmaceutical composition has a viscosity of <12 cP at 20° C. In some embodiments, the pharmaceutical composition has a viscosity of 10 cP or 11 cP at 20° C. In some embodiments, the pharmaceutical composition has a viscosity of between 6 cP to 35 cP (e.g., 6 cP, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, or 35 cP) at 20° C.

In some embodiments, the pharmaceutical composition has a percentage of higher molecular weight compounds per month at 25° C. over one month of between 0.05% (w/v) and 0.5% (w/v) (e.g., 0.05% (w/v) and 0.4% (w/v), 0.05% (w/v) and 0.3% (w/v), 0.05% (w/v) and 0.2% (w/v), 0.05% (w/v) and 0.1% (w/v), 0.1% (w/v) and 0.5% (w/v), 0.2% (w/v) and 0.5% (w/v), 0.3% (w/v) and 0.5% (w/v), or 0.4% (w/v) and 0.5% (w/v)). In some embodiments, the pharmaceutical composition has a percentage of higher molecular weight compounds per month at 25° C. over one month of about 0.3% (w/v). In some embodiments, the pharmaceutical composition has a percentage of higher molecular weight compounds per month at 37° C. over one month of between about 1% (w/v) and 10% (w/v) (e.g., 1% (w/v), 2% (w/v), 3% (w/v), 4% (w/v), 5% (w/v), 6% (w/v), 7% (w/v), 8% (w/v), 9% (w/v), or 10% (w/v)). In some embodiments, the pharmaceutical composition has a percentage of higher molecular weight compounds per month at 37° C. over one month of about 4.6% (w/v) or of about 5% (w/v). In some embodiments, the pharmaceutical composition has a percentage of higher molecular weight compounds per month at 37° C. over one month of between 4.2% (w/v) and 5.8% (w/v) or between 3.7% (w/v) and 5.5% (w/v).

In some embodiments, the pharmaceutical composition has a turbidity of between about 1 and 10 (e.g., about 1, 2, 3, 4, 5, 6, 7, 8, 9, or 10) per month at 37° C., wherein turbidity is measured as the optical density at 400 nm. In some embodiments, the pharmaceutical composition has a turbidity of about 3 or about 4 per month at 37° C. In some embodiments, the pharmaceutical composition has a turbidity between 1.4 and 4.5 per month at 37° C. or between 2.3 and 5.8 per month at 37° C.

In some embodiments, the pharmaceutical composition has an osmolality of between 100 Osm/kg H₂O and 500 Osm/kg H₂O (e.g., between 100 Osm/kg H₂O and 400 Osm/kg H₂O, 100 Osm/kg H₂O and 300 Osm/kg H₂O, 100 Osm/kg H₂O and 200 Osm/kg H₂O, 200 Osm/kg H₂O, and 500 Osm/kg H₂O, 300 Osm/kg H₂O and 500 Osm/kg H₂O, or 400 Osm/kg H₂O and 500 Osm/kg H₂O). In some embodiments, the pharmaceutical composition has an osmolality of about 245 Osm/kg H₂O.

In another aspect, the disclosure provides a pharmaceutical composition including a fusion protein, wherein the fusion protein comprises CDRs having amino acid sequences of SEQ ID NOS: 2-7, a detergent, sucrose, and sodium acetate, wherein the fusion protein is at a concentration between 120 mg/mL and 200 mg/mL, wherein the sodium acetate is at a concentration of between 25 mM and 75 mM, wherein the sucrose has a concentration of between 2% (w/v) and 15% (w/v), wherein the detergent is at a concentration between 0.01% (w/v) and 0.2% (w/v), and wherein the pharmaceutical composition has a pH of between pH 4 and pH 7.

In some embodiments, the concentration of fusion protein is about 150 mg/mL, wherein the sodium acetate concentration is about 50 mM, wherein the sucrose concentration is about 8.6% (w/v), wherein the detergent is PS-80 having a concentration of about 0.05% (w/v), and wherein the pharmaceutical composition has a pH of about 5.4.

In some embodiments, the concentration of fusion protein is about 150 mg/mL, wherein the sodium acetate concentration is about 50 mM, wherein the sucrose concentration is about 8.6% (w/v), wherein the detergent is PS-80 having a concentration of about 0.15% (w/v), and wherein the pharmaceutical composition has a pH of about 5.4.

In an aspect, the disclosure provides a pharmaceutical composition including a fusion protein, where the fusion protein includes CDRs having an amino acid sequences of SEQ ID NOS: 2-7, a detergent, and sodium acetate, where the fusion protein is at a concentration between 150 mg/mL and 200 mg/mL (e.g., between 150 mg/mL and 190 mg/mL, 150 mg/mL and 180 mg/mL, 150 mg/mL and 170 mg/mL, 150 mg/mL and 160 mg/mL, 160 mg/mL and 200 mg/mL, 170 mg/mL and 200 mg/mL, 180 mg/mL and 200 mg/mL, or 190 mg/mL and 200 mg/mL), wherein the detergent is at a concentration between 0.01% (w/v) and 0.1% (w/v) (e.g., 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), or 0.1% (w/v)), wherein the sucrose is at a concentration between 1% (w/v) and 10% (w/v) (e.g., 1% (w/v), 2% (w/v), 3% (w/v), 4% (w/v), 5% (w/v), 6% (w/v), 7% (w/v), 8% (w/v), 9% (w/v), or 10% (w/v)), wherein the sodium acetate is at a concentration between 10 mM and 50 mM (e.g., between 10 mM and 40 mM, 10 mM and 30 mM, 10 mM and 20 mM, 20 mM and 50 mM, 30 mM and 50 mM, or 40 mM and 50 mM), and wherein the pharmaceutical composition has a pH of between pH 4 and pH 7 (e.g., between pH 4 and pH 6, pH 4 and pH 5, pH 5 and pH 7, or pH 6 and pH 7). In some embodiments, the fusion protein is at a concentration of about 190 mg/mL, wherein the detergent is at a concentration of about 0.05% (w/v), wherein the sucrose is at a concentration is 4% (w/v), wherein the sodium acetate is at a concentration of about 20 mM, and wherein the pharmaceutical composition has a pH of about pH 5.4. In some embodiments, the detergent is PS80. In some embodiments, the PS80 is at a concentration of about 1.0% (w/v).

In some embodiments, the fusion protein includes CDRs having an amino acid sequences of SEQ ID NOS: 2-7, a detergent, sodium acetate, and proline, wherein the fusion protein is at a concentration between 150 mg/mL and 200 mg/mL (e.g., between 150 mg/mL and 190 mg/mL, 150 mg/mL and 180 mg/mL, 150 mg/mL and 170 mg/mL, 150 mg/mL and 160 mg/mL, 160 mg/mL and 200 mg/mL, 170 mg/mL and 200 mg/mL, 180 mg/mL and 200 mg/mL, or 190 mg/mL and 200 mg/mL), wherein the detergent is at a concentration between 0.01% (w/v) and 0.1% (w/v) (e.g., 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), or 0.1% (w/v)), wherein the sodium acetate is at a concentration between 10 mM and 50 mM (e.g., between 10 mM and 40 mM, 10 mM and 30 mM, 10 mM and 20 mM, 20 mM and 50 mM, 30 mM and 50 mM, or 40 mM and 50 mM), wherein the proline is at a concentration between 100 mM and 200 mM (e.g., between 100 mM and 180 mM, 100 mM and 160 mM, 100 mM and 140 mM, 100 mM and 120 mM, 120 mM and 200 mM, 140 mM and 200 mM, 160 mM and 200 mM, or 180 mM and 200 mM), and wherein the pharmaceutical composition has a pH of between pH 4 and pH 7 (e.g., between pH 4 and pH 6, pH 4 and pH 5, pH 5 and pH 7, or pH 6 and pH 7). In some embodiments, the fusion protein is at a concentration of about 190 mg/mL, wherein the detergent is at a concentration of about 0.05% (w/v), wherein the sodium acetate is at a concentration of about 20 mM, wherein the proline is at a concentration of about 165 mM, and wherein the pharmaceutical composition has a pH of about pH 5.4. In some embodiments, the detergent is PS80. In some embodiments, the PS80 is at a concentration of about 1.0% (w/v).

In some embodiments, the fusion protein includes a first portion including an amino acid sequence having at least 95% (e.g., at least 95%, 96%, 97%, 98%, 99%, or 100%) sequence identity to SEQ ID NO: 55 and a second portion including an amino acid sequence having at least 95% (e.g., at least 95%, 96%, 97%, 98%, 99%, or 100%) sequence identity to SEQ ID NO: 56. In some embodiments, the fusion protein has the amino acid sequence of SEQ ID NO: 1.

In some embodiments, the pharmaceutical composition is formulated as a drug product.

In another aspect, the disclosure provides a method of treating or prevention a disease or described herein including administering any one of the pharmaceutical compositions described herein to a subject in need thereof. In some embodiments, the disease is sickle cell disease. In some embodiments, the pharmaceutical composition is administered subcutaneously. In some embodiments, the pharmaceutical composition is administered via a pre-filled syringe, autoinjector or on-body device. In some embodiments, the subject is human.

In another aspect, the disclosure provides a method of developing a high concentration formulation of a fusion protein for subcutaneous administration including: i) performing an excipient screening, wherein the excipient screening includes measuring stability and viscosity of a solution including the fusion protein and one or more excipients over a period of time; ii) following step (i), measuring viscosity, turbidity, and formation rate of high molecular weight compounds in the solution of step (i) as a result of changing pH, acetate concentration, and sucrose concentration; iii) following step (ii), measuring viscosity, turbidity, and formation rate of high molecular weight compounds in the solution of step (ii) as a result of changing pH, sucrose concentration, excipient, and concentration of the fusion protein; and iv) following step (iii), performing a viscosity assessment, wherein the viscosity of the formulation is measured at a range of temperatures and a range of fusion protein concentrations.

In some embodiments, the fusion protein includes 6 CDRs having amino acid sequences of SEQ ID NOS: 2-7. In some embodiments, the fusion protein includes a first portion including an amino acid sequence having at least 95% (e.g., at least 95%, 96%, 97%, 98%, 99%, or 100%) sequence identity to SEQ ID NO: 55 and a second portion including an amino acid sequence having at least 95% (e.g., at least 95%, 96%, 97%, 98%, 99%, or 100%) sequence identity SEQ ID NO: 56. In some embodiments, the fusion protein has an amino acid sequence of SEQ ID NO: 1, or a modification thereof. In some embodiments, the modification comprises conversion of the N-terminal glutamine of the sequence of SEQ ID NO: 1 to pyro-glutamate.

In some embodiments, the high concentration formulation has a fusion protein concentration of at least 120 mg/mL. In some embodiments, the high concentration formulation has a fusion protein concentration of at least 150 mg/mL. In some embodiments high concentration formulation has a fusion protein concentration of between 120 mg/mL and 250 mg/mL (e.g., between 120 mg/mL and 250 mg/mL, 150 mg/mL and 250 mg/mL, 175 mg/mL and 250 mg/mL, 200 mg/mL and 250 mg/mL, 225 mg/mL and 250 mg/mL, 120 mg/mL and 225 mg/mL, 120 mg/mL and 200 mg/mL, 120 mg/mL and 175 mg/mL, 120 mg/mL and 150 mg/mL, or 150 mg/mL and 180 mg/mL). In some embodiments, the solution of step (i) has a fusion protein concentration of about 200 mg/mL.

In some embodiments, the measuring of step ii and step iii is performed weekly. In some embodiments, the measuring is performed for at least 4 weeks.

In some embodiments, the one or more excipients is selected from succinate, arginine, acetate, histidine, phosphate, or a combination thereof. In some embodiments, the one or more excipients is arginine. In some embodiments, the arginine is arginine hydrochloride. In some embodiments, the arginine is arginine acetate. In some embodiments, the arginine is arginine succinate. In some embodiments, the one or more excipients is succinate. In some embodiments, the one or more excipients is succinate and arginine hydrochloride. In some embodiments, the one or more excipients is acetate. In some embodiments, the one or more excipients is acetate and arginine hydrochloride. In some embodiments, the one or more excipients is acetate and arginine acetate.

In some embodiments, the solution of step (i) further includes sucrose. In some embodiments, the sucrose has a concentration of between 1% (w/v) and 15% (w/v) (e.g., 1% (w/v), 2% (w/v), 3% (w/v), 4% (w/v), 5% (w/v), 6% (w/v), 7% (w/v), 8% (w/v), 9% (w/v), 10% (w/v), 11% (w/v), 12% (w/v), 13% (w/v), 14% (w/v), or 15% (w/v)). In some embodiments, the sucrose is present at a concentration between 2% (w/v) and 10% (w/v) (e.g., 2% (w/v), 3% (w/v), 4% (w/v), 5% (w/v), 6% (w/v), 7% (w/v), 8% (w/v), 9% (w/v), or 10% (w/v)). In some embodiments, the sucrose is at a concentration between about 4% (w/v) and about 9% (w/v) (e.g., 4% (w/v), 5% (w/v), 6% (w/v), 7% (w/v), 8% (w/v), 9% (w/v), or 10% (w/v)). In some embodiments, the sucrose is at a concentration of about 4% (w/v). In some embodiments, the sucrose is present at a concentration between 8% (w/v) and 9% (w/v) (e.g., 8.1% (w/v), 8.2% (w/v), 8.3% (w/v), 8.4% (w/v), 8.5% (w/v), 8.6% (w/v), 8.7% (w/v), 8.8% (w/v), 8.9% (w/v), or 9% (w/v)). In some embodiments, the sucrose is present at a concentration of about 8.6% (w/v).

In some embodiments, the solution of step (ii) has a pH of between 4 and 7 (e.g., a pH between 4 and 6, 4 and 5, 5 and 7, or 6 and 7). In some embodiments, the solution of step (ii) has a pH of between 4.5 and 6.0 (e.g., a pH of between 4.5 and 5.5, 4.5 and 5, 5 and 6, or 5.5 and 6). In some embodiments, the solution of step (ii) has a concentration of acetate of between 10 mM and 200 mM (e.g., between 10 mM and 150 mM, 10 mM and 100 mM, 10 mM and 50 mM, 50 mM and 200 mM, 100 mM and 200 mM, or 150 mM and 200 mM). In some embodiments, the solution of step (ii) has a concentration of acetate of between 15 mM and 150 mM (e.g., between 15 mM and 80 mM, 15 mM and 60 mM, 15 mM and 40 mM, 15 mM and 20 mM, 20 mM and 100 mM, 40 mM and 100 mM, 60 mM and 100 mM, 80 mM and 100 mM, 80 mM and 150 mM, 100 mM and 150 mM, or 60 mM and 150 mM).

In some embodiments, the solution of step (ii) has a concentration of sucrose of between 1% (w/v) and 20% (w/v) (e.g., between 1% (w/v) and 15% (w/v), 1% (w/v) and 10% (w/v), 1% (w/v) and 5% (w/v), 5% (w/v) and 20% (w/v), 10% (w/v) and 20% (w/v), or 15% (w/v) and 20% (w/v)). In some embodiments, the solution of step (ii) has a concentration of sucrose of between 2% (w/v) and 10% (w/v) (e.g., 2% (w/v), 3% (w/v), 4% (w/v), 5% (w/v), 6% (w/v), 7% (w/v), 8% (w/v), 9% (w/v), or 10% (w/v)). In some embodiments, the solution of step (ii) has fusion protein concentration of about 150 mg/mL. In some embodiments, the solution of step (iii) has a pH of between 5 and 6. In some embodiments, the solution of step (iii) has a concentration of sucrose of between 0% (w/v) and 10% (w/v) (e.g., 1% (w/v), 2% (w/v), 3% (w/v), 4% (w/v), 5% (w/v), 6% (w/v), 7% (w/v), 8% (w/v), 9% (w/v), or 10% (w/v)). In some embodiments, the solution of step (iii) has a concentration of sucrose of between 0% (w/v) and 5% (w/v) (e.g., 1% (w/v), 2% (w/v), 3% (w/v), 4% (w/v), or 5% (w/v)). In some embodiments, the solution of step (iii) has a fusion protein concentration of between about 100 mg/mL and 300 mg/mL (e.g., between 100 mg/mL and 250 mg/mL, 100 mg/mL and 200 mg/mL, 100 mg/mL and 150 mg/mL, 150 mg/mL and 300 mg/mL, 200 mg/mL and 300 mg/mL, or 250 mg/mL and 300 mg/mL). In some embodiments, the solution of step (ii) has a fusion protein concentration of between about 150 mg/mL and 230 mg/mL (e.g., 150 mg/mL and 200 mg/mL, 150 mg/mL and 175 mg/mL, 175 mg/mL and 230 mg/mL, 200 mg/mL and 230 mg/mL, or 220 mg/mL and 230 mg/mL).

In some embodiments, the formulation has a viscosity of between 5 cP and 15 cP (e.g., 5 cP, 6 cP, 7 cP, 8 cP, 9 cP, 10 cP, 11 cP, 12 cP, 13 cP, 14 cP, or 15 cP) at 20° C. In some embodiments, the formulation has a viscosity about 10 cP or 11 cP at 20° C. In some embodiments, the formulation has a percentage of higher molecular weight compounds per month at 25° C. over one month of between 0.05% (w/v) and 0.5% (w/v) (e.g., between 0.05% (w/v) and 0.4% (w/v), 0.05% and 0.3% (w/v), 0.05% and 0.2% (w/v), 0.05% (w/v) and 0.1% (w/v), 0.1% (w/v) and 0.5% (w/v), 0.2% (w/v) and 0.5% (w/v), 0.3% (w/v) and 0.5% (w/v), or 0.4% (w/v) and 0.5% (w/v)). In some embodiments, the formulation has a percentage of higher molecular weight compounds per month at 25° C. over one month of about 0.2% (w/v). In some embodiments, the formulation has a percentage of higher molecular weight compounds per month at 37° C. over one month of between about 1% (w/v) and 10% (w/v) (e.g., 1% (w/v), 2% (w/v), 3% (w/v), 4% (w/v), 5% (w/v), 6% (w/v), 7% (w/v), 8% (w/v), 9% (w/v), or 10% (w/v)). In some embodiments, the formulation has a percentage of higher molecular weight compounds per month at 37° C. over one month of about 5% (w/v). In some embodiments, the formulation has a turbidity of between about 1 and 10 (e.g., 1, 2, 3, 4, 5, 6, 7, 8, 9, or 10) per month at 37° C., wherein turbidity is measured as the optical density at 400 nm. In some embodiments, the formulation has a turbidity of about 3 per month at 37° C.

In another aspect, the disclosure provides a bispecific construct that binds properdin and human serum albumin, the bispecific construct including the amino acid sequence of SEQ ID NO:1, or a modification thereof. The bispecific construct of claim 117, wherein the modification includes conversion of the N-terminal glutamine of the amino acid sequence of SEQ ID NO:1 to pyro-glutamate. In another embodiment, the bispecific construct consists of the amino acid sequence of SEQ ID NO:1 where the N-terminal glutamine has been converted to pyro-glutamate.

### BRIEF DESCRIPTION OF THE DRAWINGS

FIG. 1A to FIG. 1C are graphs showing the effect various buffering excipients (FIG. 1A), the presence of proline (FIG. 1B), and the sucrose concentration (FIG. 1C) have on viscosity of a solution viscosity of a solution having 200 mg/mL of the fusion protein over a range of temperatures.

FIG. 2A and FIG. 2B are graphs showing the percentage of higher molecular weight compounds in solutions having various buffering excipients (FIG. 2A) or sucrose concentrations or presence of proline (FIG. 2B) over a period of 4 weeks at 37° C.

FIG. 3A and FIG. 3B are photographs showing the turbidity in solutions having various buffering excipients (FIG. 2A) or sucrose concentrations or presence of proline (FIG. 2B) over a period of 4 weeks at 37° C.

FIG. 4 is a graph of showing the measurement of the turbidity of the solution over a 4 week period for different sucrose concentrations.

FIG. 5A and FIG. 5B are graphs showing the measurement of the percentage of higher molecular weight compounds at 37° C. (FIG. 5A) or 25° C. (FIG. 5B) over a period of 3 months in solutions having different concentrations of acetate.

FIG. 6A and FIG. 6B are graphs showing the measurement of the percentage of acid present in solution at 37° C. (FIG. 6A) or 25° C. (FIG. 6B) over a period of 3 months in solutions having different concentrations of acetate.

FIG. 7A and FIG. 7B are graphs showing the measurement of the percentage of base present in solution at 37° C. (FIG. 7A) or 25° C. (FIG. 7B) over a period of 3 months in solutions having different concentrations of acetate.

FIG. 8A and FIG. 8B are graphs showing the measurement of the turbidity of the solution at 37° C. (FIG. 5A) or 25° C. (FIG. 5B) over a period of 3 months in solutions having different concentrations of acetate.

FIG. 9 shows a series of graphs showing the predicting effects of acetate concentration, sucrose concentration, and pH on osmolality, viscosity at 25° C., and viscosity at 20° C. of a solution having a concentration of 150 mg/mL of a polypeptide having the amino acid sequence of SEQ ID NO. 1.

FIG. 10A and FIG. 10B show a series of graphs showing the predicting effects of acetate concentration, sucrose concentration, and pH at 37° C. (FIG. 10A) and 25° C. (FIG. 10B) on the rate of higher molecular weight compound formation, turbidity, the rate of acid formation, and the rate of base formation of a solution having a concentration of 150 mg/mL of a polypeptide having the amino acid sequence of SEQ ID NO. 1.

FIG. 11A and FIG. 11B are graphs showing the measurement of the percentage of higher molecular weight compounds at 37° C. (FIG. 11A) or 25° C. (FIG. 11B) over a period of 1 month in solutions having different concentrations of acetate.

FIG. 12A and FIG. 12B are graphs showing the measurement of the percentage of acid present in solution at 37° C. (FIG. 12A) or 25° C. (FIG. 12B) over a period of 1 month in solutions having different concentrations of acetate.

FIG. 13A and FIG. 13B are graphs showing the measurement of the percentage of base present in solution at 37° C. (FIG. 13A) or 25° C. (FIG. 13B) over a period of 1 month in solutions having different concentrations of acetate.

FIG. 14A and FIG. 14B are graphs showing the measurement of the turbidity of the solution at 37° C. (FIG. 14A) or 25° C. (FIG. 14B) over a period of 1 month in solutions having different concentrations of acetate.

FIG. 15 shows a series of graphs showing the predicting effects of polypeptide concentration, proline concentration, sucrose concentration, and pH on the rate of higher molecular weight compound formation, turbidity, the rate of acid formation, and the rate of base formation of a solution.

FIG. 16A and FIG. 16B show a series of graphs showing the predicting effects of polypeptide concentration, proline concentration, sucrose concentration, and pH at 37° C. (FIG. 16A) and 25° C. (FIG. 16B) on the rate of higher molecular weight compound formation, turbidity, the rate of acid formation, and the rate of base formation of a solution.

FIG. 17 is a graph showing the effect of sucrose concentration on the viscosity of solutions having at least 200 mg/mL polypeptide (fusion protein) concentration.

### DEFINITIONS

To facilitate the understanding of this disclosure, a number of terms are defined below. Terms defined herein have meanings as commonly understood by a person of ordinary skill in the areas relevant to the disclosure. Terms such as “a,” “an” and “the” are not intended to refer to only a singular entity, but include the general class of which a specific example may be used for illustration. The terminology herein is used to describe specific embodiments, but their usage does not limit the disclosure, except as outlined in the claims.

As used herein, the term “about” refers to a value that is within 10% above or below the value being described.

As used herein, any values provided in a range of values include both the upper and lower bounds, and any values contained within the upper and lower bounds.

The term “antibody” as used herein includes whole antibodies and any antigen binding fragment (i.e., “antigen-binding portion”) or single chain version thereof. An “antibody” refers to a glycoprotein, for example, comprising at least two heavy (H) chains and two light (L) chains inter-connected by disulfide bonds, or an antigen binding portion thereof. The terms “heavy chain” and “light chain,” as used herein, refer to any immunoglobulin (“Ig”) polypeptide having sufficient variable domain sequence to confer specificity for a target antigen. Each heavy chain is comprised of a heavy chain variable region (abbreviated herein as V_{H}) and a heavy chain constant region. The heavy chain constant region is comprised of three domains—CH1, CH2 and CH3. Each light chain is comprised of a light chain variable region (abbreviated herein as V_{L}) and a light chain constant region. The light chain constant region is comprised of one domain, CL. Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody (or simply “antibody fragment”), as used herein, refers to one or more fragments or portions of an antibody that retain the ability to specifically bind to an antigen. Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs, which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (sFv); Bird, R. et al., Science 242:423-6, 1988; Huston, J. et al., Proc. Natl. Acad. Sci. USA, 85:5879-83, 1988). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

An antibody, immunoglobulin, or immunologically functional immunoglobulin fragment, or the engineered polypeptides or fusion proteins disclosed herein, are said to “specifically” bind an antigen when the molecule preferentially recognizes its antigen target in a complex mixture of proteins and/or macromolecules. The term “specifically binds,” as used herein, refers to the ability of an antibody, immunoglobulin, or immunologically functional immunoglobulin fragment, or an engineered polypeptide or fusion protein of the disclosure, to bind to an antigen containing an epitope with an K_{D} of at least about 10⁻⁶ M, 10⁻⁷ M, 10⁻⁸ M, 10⁻⁹ M, 10⁻¹⁰ M, 10⁻¹¹ M, 10⁻¹² M, or less, and/or to bind to an epitope with an affinity that is at least two-fold greater than its affinity for a nonspecific antigen.

A “complement-mediated disorder” as used herein refers to a disorder caused, directly or indirectly, by mis-regulation of the complement pathway, e.g., activation or suppression of the complement pathway, or a disorder that is mediated, directly or indirectly, by one or more components of the complement pathway, or a product generated by the complement pathway. The term also refers to a disorder that is exacerbated by one or more components of the complement pathway, or a product generated by the complement pathway.

As used herein, a “drug product” or “DP” refers to a pharmaceutical composition in its final configuration such that it is ready to be administered to a subject (e.g., in final vial configuration). The concentration of polypeptide in a DP may be the concentration at which it is to be administered to the subject.

As used herein, the term “effective amount,” refers to a quantity of a pharmaceutical composition sufficient to, when administered to the subject, for example a human subject, effect beneficial or desired results, such as clinical results. The quantity of a given composition described herein that will correspond to such beneficial or desired results depends on various factors, such as, for example, the given agent, the pharmaceutical formulation, the route of administration, the identity of the subject (e.g., age, sex, weight) being treated, and the like.

The term “fused to” as used herein refers to a polypeptide engineered by combining more than one sequence, typically by cloning one sequence, e.g., a coding sequence, into an expression vector in frame with one or more second coding sequence(s) such that the two (or more) coding sequences are transcribed and translated into a single continuous polypeptide. In addition to being made by recombinant technology, parts of a polypeptide can be “fused to” each other by means of chemical reaction, or other means known in the art for making custom polypeptides.

The term “heavy chain antibody,” as used herein, refers to an antibody that lacks the light chain(s) found in conventional antibodies.

The term “human antibody,” as used herein, refers to an Ig that is used, for example, by the immune system to bind and neutralize pathogens. The term includes antibodies having variable and constant regions substantially corresponding to human germline Ig sequences. In some embodiments, human antibodies are produced in non-human mammals, including, but not limited to, rodents, such as mice and rats, and lagomorphs, such as rabbits. In other embodiments, human antibodies are produced in hybridoma cells. In still other embodiments, human antibodies are produced recombinantly. As used herein, human antibodies include all or a portion of an antibody, including, for example, heavy and light chains, variable regions, constant regions, proteolytic fragments, complementarity determining regions (CDRs), and other functional fragments.

The term “VHH domain” refers to a variable domain present in naturally occurring heavy chain antibodies (e.g., a “VHH antibody” or “VHH single chain antibody”) to distinguish them from the heavy chain variable domains that are present in conventional four chain antibodies (referred to herein as “VH domains”) and from the light chain variable domains that present in conventional four chain antibodies (referred to herein as “VL domains”). VHH antibodies are produced by certain camelid species, e.g., camels and llamas. Camelids immunized against a particular antigen will produce single chain antibodies (a single heavy chain) that bind to the antigen. Single domain, heavy chain variable domain sequences from a heavy chain antibody may be referred to as VHH or V_{H}H antibodies, VHH or V_{H}H antibody fragments, or VHH or V_{H}H domains.

The term “peptide linker,” as used herein, refers to one or more amino acid residues inserted or included between the polypeptides of a fusion protein. The peptide linker can be, for example, inserted or included at the transition between the polypeptides of the fusion protein at the sequence level. The identity and sequence of amino acid residues in the linker may vary depending on the desired secondary structure. For example, glycine, serine and alanine are useful for linkers having maximum flexibility. Any amino acid residue can be considered as a linker in combination with one or more other amino acid residues, which may be the same as or different from the first amino acid residue, to construct larger peptide linkers as necessary depending on the desired properties.

The term “bispecific” refers to a fusion protein that is capable of binding two antigens. The term “multivalent fusion protein” means a fusion protein comprising two or more antigen binding sites. A “multi-specific fusion protein” is a fusion protein that is capable of binding two or more related or unrelated targets. Conventional four-chain antibodies are multivalent, but each arm binds to the same antigen and, thus, are not bispecific. VHH antibodies are monovalent. Bispecific antibodies can be engineered, for example, by fusing two or more monovalent VHH antibodies (or VHH variable domain fragments or other functional fragments), with or without a linker, such that the fusion protein is multivalent. If the fused variable domains specifically bind different antigens, then the fusion protein would be bispecific.

The term “vector,” as used herein, refers to any molecule (e.g., nucleic acid, plasmid or virus) that is used to transfer coding information to an expression system (e.g., a host cell or in vitro expression system). One type of vector is a “plasmid,” which refers to a circular double-stranded DNA (dsDNA) molecule into which additional DNA segments can be inserted. Another type of vector is a viral vector, wherein additional DNA segments can be inserted into a viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of coding sequences to which they are operatively linked. Such vectors are referred to herein as “expression vectors.”

The term “operably linked,” as used herein, refers to an arrangement of flanking sequences wherein the flanking sequences are configured or assembled to perform a desired function. Thus, a flanking sequence operably linked to a coding sequence may be capable of effecting the replication, transcription, and/or translation of the coding sequence. A coding sequence is operably linked to a promoter, for example, where the promoter is capable of directing transcription of that coding sequence. A flanking sequence need not be contiguous with the coding sequence to be considered operably linked, so long as it functions correctly.

The term “host cell,” as used herein, refers to a cell into which an expression vector has been introduced. A host cell is intended to refer not only to the particular subject cell, but also to the progeny of such a cell. Because certain modifications may occur in succeeding generations due to either mutation or environmental influences, such progeny may not be, in fact, identical to the parent cell, but such cells are still included within the scope of the term “host cell” as used herein. A wide variety of host cell expression systems can be used to express the fusion protein of the disclosure, including bacterial, yeast, baculoviral, and mammalian expression systems (as well as phage display expression systems).

The terms “patient” and “subject” as used herein include human and animal subjects.

The terms “pharmaceutical composition” or “therapeutic composition,” as used herein, refer to a compound or composition capable of inducing a desired therapeutic effect when administered to a patient.

The term “pharmaceutically acceptable carrier” or “physiologically acceptable carrier,” as used herein, refers to one or more formulation materials suitable for accomplishing or enhancing the delivery of the fusion protein of the disclosure.

As used herein, the term “pharmaceutically acceptable salt,” represents those salts that are suitable for use in the treatment of humans without undue toxicity. Pharmaceutically acceptable salts are known in the art (Berge, S. et al., J. Pharm. Sci., 66:1-19, 1977; Pharmaceutical Salts: Properties, Selection, and Use, (Eds. P. H. Stahl and C. G. Wermuth), Wiley-VCH, 2008). The salts can be prepared, for example, in situ during the final isolation and purification of the compounds described herein or separately by reacting the free base group with a suitable organic acid. Representative acid addition salts include, without limitation, acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include, without limitation, sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, L-α-phosphatidylethanolamine, bis(2-ethylhexyl)amine, soy Lecithin and the like. Representative amino acid salts include lysine, arginine, glycine, histidine, and the like. One of skill in the art will recognize that any mention of a drug compound includes within its scope the pharmaceutically acceptable salts of the indicated drug compound.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences encoded by the germline genes, but can also include subsequent rearrangements and mutations that occur, for example, during antibody maturation. The variable region contains the antigen binding domain, which is encoded by various genes that rearrange to form an antibody specific for a foreign antigen (Lonberg, N., Nat. Biotechnol., 23:1117-25, 2005). In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

The terms “treatment” or “treat,” as used herein, refer to both therapeutic treatment and prophylactic or preventative measures. Those in need of treatment include those having a disease or condition as well as those at risk of having the disease or condition or those in which the disease or condition is to be prevented.

As used herein, a “therapeutically effective” amount of, for example, a fusion protein described herein, refers to an amount or dosage sufficient to produce a desired therapeutic result. A therapeutically effective amount is an amount is an amount that, when administered, results in a decrease in severity of disease symptoms (e.g., an increase in frequency and duration of disease symptom free periods or a prevention of impairment or disability due to the disease affliction) or that is sufficient to inhibit, for some period of time, one or more of the clinically defined pathological processes associated with the condition being treated. The therapeutically effective amount may vary depending on a variety of factors and conditions related to the patient being treated and the severity of the disorder.

### DETAILED DESCRIPTION

The instant disclosure is based in part on the optimization of pharmaceutical compositions that allow for fusion proteins to be present at high concentrations, which is desired in in the development of compositions for subcutaneous administration. A particular need arises for high concentration formulations of pharmaceutical compositions comprising biomolecules, e.g., enzymes, antibodies, VHH antibodies, bispecific VHH antibodies and bispecific VHH antibody fragments. The pharmaceutical composition described herein include a fusion protein (e.g., a VHH bispecific antibody comprising variable domains from two different camelid antibodies joined by a flexible linker), polysorbate, and a buffering agent. These formulations have been optimized in view of viscosity and stability such the fusion protein is present at a concentration of at least 150 mg/mL.

### Pharmaceutical Compositions

The fusion proteins described herein can be incorporated into pharmaceutical compositions suitable for administration to a subject. Typically, the pharmaceutical composition includes a fusion protein and one or more pharmaceutically acceptable carriers or excipients.

The pharmaceutical compositions described herein included a fusion protein that is present at a concentration of at least 150 mg/mL. For example, the concentration of the fusion protein in the pharmaceutical composition may be between 150 mg/mL and 200 mg/mL (e.g., between 150 mg/mL and 190 mg/mL, 150 mg/mL and 180 mg/mL, 150 mg/mL and 170 mg/mL, 150 mg/mL and 160 mg/mL, 160 mg/mL and 200 mg/mL, 170 mg/mL and 200 mg/mL, 180 mg/mL and 200 mg/mL, or 190 mg/mL and 200 mg/mL). In some embodiments, the concentration of the fusion protein is between 170 mg/mL and 200 mg/mL (e.g., 170 mg/mL, 171 mg/mL, 172 mg/mL, 173 mg/mL, 174 mg/mL, 175 mg/mL, 176 mg/mL, 177 mg/mL, 178 mg/mL, 179 mg/mL, 180 mg/mL, 181 mg/mL, 182 mg/mL, 183 mg/mL, 184 mg/mL, 185 mg/mL, 186 mg/mL, 187 mg/mL, 188 mg/mL, 189 mg/mL, 190 mg/mL, 191 mg/mL, 192 mg/mL, 193 mg/mL, 194 mg/mL, 195 mg/mL, 196 mg/mL, 197 mg/mL, 198 mg/mL, 199 mg/mL, or 200 mg/mL). In some embodiments, the concentration of the fusion protein is about 190 mg/mL. In other embodiments, the concentration of the fusion protein is about 150 mg/mL. The methods and composition described herein can allow for even higher concentrations, e.g., up to 250 mg/mL and higher.

The pharmaceutical composition described herein may include a detergent. The pharmaceutical composition may have a concentration of detergent of between 0.01% (w/v) and 1.5% (w/v) (e.g., between 0.01% (w/v) and 0.4% (w/v), 0.01% (w/v) and 0.3% (w/v), 0.01% (w/v) and 0.2% (w/v), 0.01% (w/v) and 0.1 (w/v), 0.01% (w/v) and 0.05% (w/v), 0.05% (w/v) and 0.5% (w/v), 0.1% (w/v) and 0.5% (w/v), 0.2% (w/v) and 0.5% (w/v), 0.3% (w/v) and 0.5% (w/v), or 0.4% (w/v) and 0.5% (w/v)). In some embodiments, the detergent is a concentration is between 0.01% (w/v) and 1% (w/v) (e.g., between 0.01% (w/v) and 0.9% (w/v), 0.01% (w/v) and 0.5% (w/v), 0.01% (w/v) and 0.1% (w/v), 0.01% (w/v) and 0.05% (w/v), 0.05% (w/v) and 1% (w/v), 0.1 (w/v) and 0.1% (w/v), or 0.5% and 1% (w/v)). In some embodiments, the detergent is a concentration is between 0.01% (w/v) and 0.1% (w/v) (e.g., 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), or 0.1% (w/v)). In certain embodiments, the detergent is at a concentration of about 0.05% (w/v). In other embodiments, the detergent is at a concentration of about 0.1% (w/v). The detergent may be any detergent known to one skilled in the art. For example, the pharmaceutical compositions described herein may include polysorbate, TRITON® X-100, digitonin, saponin, n-dodecyl-β-D-maltoside, or any combination thereof. In some embodiments, the polysorbate may be polysorbate 80 (PS80) or polysorbate 20.

The pharmaceutical compositions described herein include one or more buffering agents. The buffering agent may be any buffering agent known in the art. For example, the buffering agent may be acetate, succinate, histidine, phosphate, or a combination thereof. The buffering agent may be present at any concentration that allows for buffering of the pharmaceutical composition. In some embodiments, the buffering agent has as concentration of between about 10 mM and 200 mM (e.g., between 10 mM and 150 mM, 10 mM and 100 mM, 10 mM and 50 mM, 50 mM and 200 mM, 100 mM and 200 mM, or 150 mM and 200 mM). For example, the buffering agent has a concentration of between about 100 mM and 200 mM (e.g., 100 mM and 180 mM, 100 mM and 160 mM, 100 mM and 140 mM, 100 mM and 120 mM, 120 mM and 200 mM, 140 mM and 200 mM, 160 mM and 200 mM, or 180 mM and 200 mM).

In some embodiments, the pharmaceutical composition includes an acetate buffering agent. For example, the acetate is sodium acetate. In some embodiments, the sodium acetate is present in the pharmaceutical composition at concentration between 10 mM and 150 mM (e.g., between 10 mM and 140 mM, 10 mM and 130 mM, 10 mM and 120 mM, 10 mM and 100 mM, 10 mM and 75 mM, 10 mM and 50 mM, 20 mM and 150 mM, 30 mM and 150 mM, 75 mM and 150 mM, 100 mM and 150 mM or 40 mM and 150 mM). In some embodiments, the concentration of the sodium acetate is between about 15 mM and 100 mM (e.g., between about 15 mM and 80 mM, 15 mM and 60 mM, 15 mM and 40 mM, 15 mM and 20 mM, 20 mM and 100 mM, 40 mM and 100 mM, 60 mM and 100 mM, or 80 mM and 100 mM. In some embodiments, the concentration of the sodium acetate is about 50 mM.

In some embodiments, the pharmaceutical composition includes an amino acid stabilizer, e.g., proline or arginine. In some embodiments, the amino acid stabilizer (e.g., proline) is present in the pharmaceutical composition at concentration between 100 mM and 200 mM (e.g., between 100 mM and 190 mM, 100 mM and 180 mM, 100 mM and 170 mM, 140 mM and 200 mM, 150 mM and 200 mM, or 160 mM and 200 mM, 110 mM and 190 mM, 120 mM and 180 mM, 130 mM and 170 mM, 140 mM and 180 mM, 150 mM and 170 mM or 160 mM and 170 mM). In particular embodiments, the concentration of proline is about 165 mM. In some embodiments, the arginine is arginine acetate, arginine-succinate, or arginine hydrochloride

The pharmaceutical compositions described herein may include a tonicity agent. The tonicity agent may be any tonicity agent known in the art. For example, the tonicity agent may be a sugar, an amino acid, or a salt. The sugar may be sucrose, glucose, glycerol, or trehalose. In some embodiments, the sugar is sucrose. The sucrose may have a concentration of between 1% (w/v) and about 15% (w/v) (e.g., 1% (w/v), 2% (w/v), 3% (w/v), 4% (w/v), 5% (w/v), 6% (w/v), 7% (w/v), 8% (w/v), 9% (w/v), 10% (w/v), 11% (w/v), 12% (w/v), 13% (w/v), 14% (w/v), or 15% (w/v)). For example, the sucrose may have a concentration between about 4% (w/v) and about 9% (w/v) (e.g., 4% (w/v), 5% (w/v), 6% (w/v), 7% (w/v), 8% (w/v), 9% (w/v), or 10% (w/v)). In some embodiments, the sucrose is at a concentration of about 4% (w/v). In some embodiments, the sucrose is present at a concentration between 8% (w/v) and 9% (w/v) (e.g., 8.1% (w/v), 8.2% (w/v), 8.3% (w/v), 8.4% (w/v), 8.5% (w/v), 8.6% (w/v), 8.7% (w/v), 8.8% (w/v), 8.9% (w/v), or 9% (w/v)).

The pH of the pharmaceutical composition may be between pH 3 and pH 8 (e.g., pH 3, pH 4, pH 5, pH 6, pH 7, or pH 8). In some embodiments, the pharmaceutical composition has a pH of between pH 4 and pH 7 (e.g., pH 4, pH 4.5, pH 5, pH 5.5, pH 6, pH 6.5, or pH 7). For example, the pH of the pharmaceutical composition may be pH 5.4.

The pharmaceutical composition may be formulated such that the pharmaceutical composition has a viscosity of <17 cP at 20° C. In some embodiments, the pharmaceutical composition may be formulated such that the pharmaceutical composition has a viscosity of <12 cP at 20° C. For example, the viscosity may be 10 or 11 cP at 20° C. In some embodiments, the pharmaceutical composition has a viscosity of between 6 cP to 35 cP (e.g., 6 cP, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, or 35 cP) at 20° C.

In some embodiments, the pharmaceutical composition may be formulated such that the pharmaceutical composition has a percentage of higher molecular weight compounds per month at 25° C. over one month of between 0.05% (w/v) and 0.5% (w/v) (e.g., 0.05% (w/v) and 0.4% (w/v), 0.05% (w/v) and 0.3% (w/v), 0.05% (w/v) and 0.2% (w/v), 0.05% (w/v) and 0.1% (w/v), 0.1% (w/v) and 0.5% (w/v), 0.2% (w/v) and 0.5% (w/v), 0.3% (w/v) and 0.5% (w/v), or 0.4% (w/v) and 0.5% (w/v)). In some embodiments, the pharmaceutical composition has a percentage of higher molecular weight compounds per month at 25° C. over one month of about 0.2% (w/v) or in the range between 0.1 and 0.2 or between 0.1 and 0.3. The pharmaceutical composition may have a percentage of higher molecular weight compounds per month at 37° C. over one month of between about 1% (w/v) and 10% (w/v) (e.g., 1% (w/v), 2% (w/v), 3% (w/v), 4% (w/v), 5% (w/v), 6% (w/v), 7% (w/v), 8% (w/v), 9% (w/v), or 10% (w/v), or between 4.2% (w/v) and 5.8% (w/v), or between about 3.7% (w/v) and about 5.5% (w/v)). For example, the pharmaceutical composition may have a percentage of higher molecular weight compounds per month at 37° C. over one month of about 5% (w/v) or about 4.6% (w/v).

The pharmaceutical composition may have a turbidity of between about 1 and 10 (e.g., 1, 2, 3, 4, 5, 6, 7, 8, 9 and 10) with respect to optical density (OD) at 400 nm after one month when stored 37° C. For example, the pharmaceutical composition may have a turbidity of about 3.0 or 4.0 per month at 37° C., as measured by OD 400 or the pharmaceutical composition may have a turbidity of between 1.4 and 4.5 or between 2.3 and 5.8 per month at 37° C., as measured by OD 400.

In some embodiments, the pharmaceutical composition has an osmolality of between 100 Osm/kg H₂O and 500 Osm/kg H₂O (e.g., between 100 Osm/kg H₂O and 400 Osm/kg H₂O, 100 Osm/kg H₂O and 300 Osm/kg H₂O, 100 Osm/kg H₂O and 200 Osm/kg H₂O, 200 Osm/kg H₂O and 500 Osm/kg H₂O, 300 Osm/kg H₂O, and 500 Osm/kg H₂O, or 400 Osm/kg H₂O and 500 Osm/kg H₂O). In some embodiments, the pharmaceutical composition has an osmolality of about 245 Osm/kg H₂O.

### Engineered Fusion Protein

Described herein are fusion proteins and formulations thereof. In some embodiments, the fusion protein is a bispecific antibody where two antigen binding polypeptides are linked (e.g., by a linker such as the linker). Such bispecific constructs may include an anti-properdin binding polypeptide (e.g., a monovalent VHH antibody, or VHH variable domain) connected by a linker to a second polypeptide (e.g., a second monovalent antibody or VHH variable domain). The second polypeptide can, for example, enhance in vivo stability of the bispecific construct, target a different therapeutic target, or place two antigens in close proximity (e.g., thereby targeting the first bound antigen to the second bound antigen). In some embodiments, the second polypeptide is an albumin binding molecule, an albumin binding peptide, or an anti-albumin antibody (e.g., a monovalent antibody), or a modified form thereof (e.g., the variable domain of a llama antibody that specifically binds to human serum albumin). In addition to the present disclosure, albumin binding peptides are known in the art (WO 2007/106120 (see Tables 1 to 9); Dennis, M. et al., J. Biol. Chem., 277:35035-43, 2002; the disclosures of which are hereby incorporated by reference).

The antibodies described herein can inhibit, for example, properdin binding to C3b, C3Bb, and C3bBb. Inhibition of properdin leads to reduced alternative pathway complement activation, indicating a therapeutic benefit for patients afflicted with a disease of alternative pathway dysregulation wherein the alternative pathway is hyper-activated.

Anti-properdin antibodies described herein can be produced by using full-length properdin, properdin polypeptides, and/or using antigenic properdin epitope-bearing peptides, for example, a fragment of the properdin polypeptide. Properdin peptides and polypeptides can be isolated and used to generate antibodies as natural polypeptides, recombinant or synthetic recombinant polypeptides. All antigens useful for producing anti-properdin antibodies can be used to generate monovalent antibodies. Suitable monovalent antibody formats, and methods for producing them, are known in the art (WO 2007/048037 and WO 2007/059782, the entire contents of which are incorporated herein by reference).

The anti-properdin antibody may be a monoclonal antibody or derived from a monoclonal antibody. Suitable monoclonal antibodies to selected antigens may be prepared by known techniques (“Monoclonal Antibodies: A manual of techniques,” Zola (CRC Press, 1988); “Monoclonal Hybridoma Antibodies: Techniques and Applications,” Hurrell (CRC Press, 1982), the entire contents of which are incorporated herein by reference).

In other embodiments, the antibody may be a single-domain antibody, such as a VHH. Such antibodies exist naturally in camelids and sharks (Saerens, D. et al., Curr. Opin. Pharmacol., 8:600-8, 2008). Camelid antibodies are described in, for example, U.S. Pat. Nos. 5,759,808; 5,800,988; 5,840,526; 5,874,541; 6,005,079; and 6,015,695, the entire contents of each of which are incorporated herein by reference. The cloned and isolated VHH domain is a stable polypeptide that features the full antigen-binding capacity of the original heavy-chain antibody. VHH domains, with their unique structural and functional properties, combine the advantages of conventional antibodies (high target specificity, high target affinity and low inherent toxicity) with important features of small molecule drugs (the ability to inhibit enzymes and access receptor clefts). Furthermore, they are stable, have the potential to be administered by means other than injection, are easier to manufacture, and can be humanized (U.S. Pat. Nos. 5,840,526; 5,874,541; 6,005,079, 6,765,087; EP 1589107; WO 97/34103; WO 97/49805; U.S. Pat. Nos. 5,800,988; 5,874,541 and 6,015,695, the entire contents of each of which are incorporated herein by reference).

The anti-properdin component of the bispecific antibodies described herein CDR sequences including CDR-H1 having an amino acid sequence that is at least 90% identical to GRISSIIHMA (SEQ ID NO: 2); CDR-H2 having an amino acid sequence that is at least 90% identical (e.g., at least 95% 96%, 97%, 98%, 99%, or 100%) to RVGTTVYADSVKG (SEQ ID NO: 3); and having an amino acid sequence that is at least 90% identical (e.g., at least 95%, 96%, 97%, 98%, 99%, or 100%) to LQYEKHGGADY (SEQ ID NO: 4). The bispecific antibodies described herein may include CDR-H1 having an amino acid sequence of GRISSIIHMA (SEQ ID NO: 2); CDR-H2 having an amino acid sequence of RVGTTVYADSVKG (SEQ ID NO: 3); and CDR-H3 having an amino acid sequence of LQYEKHGGADY (SEQ ID NO: 4).

Furthermore, the engineered fusion proteins described herein can specifically bind serum albumin in such a way that, when the engineered protein is bound to or otherwise associated with a serum albumin molecule, the binding of the serum albumin molecule to FcRn is not significantly reduced or inhibited as compared to the binding of the serum albumin molecule to FcRn when the polypeptide is not bound thereto. In this embodiment, “not significantly reduced or inhibited” means that the binding affinity for serum albumin to FcRn (as measured using a suitable assay, such as, for example, SPR) is not reduced by more than 50%, or by more than 30%, or by more than 10%, or by more than 5%, or not reduced at all. In this embodiment, “not significantly reduced or inhibited” also means that the half-life of the serum albumin molecule is not significantly reduced. In particular, the engineered polypeptides can bind to amino acid residues on serum albumin that are not involved in binding of serum albumin to FcRn. More particularly, engineered polypeptides can bind to amino acid residues or sequences of serum albumin that do not form part of domain III of serum albumin, e.g., engineered polypeptides that are capable of binding to amino acid residues or sequences of serum albumin that form part of domain I and/or domain II.

The anti-albumin component of the bispecific antibodies described herein can comprise CDR sequences including CDR-H1 having an amino acid sequence that is at least 87% identical to GRPVSNYA (SEQ ID NO: 5); CDR-H2 having an amino acid sequence that is at least 87% identical to INWQKTAT (SEQ ID NO: 6); and having an amino acid sequence that is at least 90% identical (e.g., at least 95%, 96%, 97%, 98%, 99%, or 100%) to AAVFRVVAPKTQYDYDY (SEQ ID NO: 7). The bispecific antibodies described herein may include CDR-H1 having an amino acid sequence of GRPVSNYA (SEQ ID NO: 5); CDR-H2 having an amino acid sequence of INWQKTAT (SEQ ID NO: 6); and CDR-H3 having an amino acid sequence of AAVFRVVAPKTQYDYDY (SEQ ID NO: 7).

In some embodiments, the fusion protein includes an anti-properdin binding portion and an anti-albumin binding portion. In some embodiments, where the anti-properdin binding domain has an exposed N-terminus, the N-terminal glutamine can convert into the cyclized pyro-glutamate. Such modifications are known in the art (see, e.g., Liu et al., The Journal of Biological Chemistry 286(13:11211-11217, 2011). The portion encoding the anti-properdin binding portion may have at least 90% (e.g., 95%, 96%, 97%, 98%, 99%, or 100%) identity to the amino acid sequence of:

(SEQ ID NO: 55)
QVQLVESGGG LVKPGGSLRL SCAASGRPVS
NYAAAWFRQA PGKEREFVSA INWQKTATYA
DSVKGRFTIS RDNAKNSLYL QMNSLRAEDT
AVYYCAAVFR VVAPKTQYDY DYWGQGTLVT
VSS.

In some embodiments, the anti-properdin binding portion of the fusion protein includes SEQ ID NO: 55. The portion encoding the anti-albumin binding portion may have at least 90% (e.g., 95%, 96%, 97%, 98%, 99%, or 100%) identity to the amino acid sequence of:

(SEQ ID NO: 56)
VQ LLESGGGLVQ PGGSLALSCA ASGRISSIIH
MAWFRQAPGK ERELVSEISR VGTTVYADSV
KGRFTISRDN SKNTLYLQMN SLKPEDTAVY
YCNALQYEKH GGADYWGQGT LVTVSS.

In some embodiments, the anti-properdin binding portion of the fusion protein includes SEQ ID NO: 56.

In some embodiments, the fusion protein is encoded by a nucleic acid sequence having at least 80% (e.g., at least 81%, 82%, 83%, 84%, 85%, 86%, 87%, 88%, 89%, 90%, 91%, 92%, 93%, 94%, 95%, 96%, 97%, 98%, 99%, or 100%) identity to the nucleic sequence of:

(SEQ ID NO: 57)
CAGGTGCAGCTGGTGGAAAGCGGCGGAGGCCTGGTCAAGCCTGGCGGCAGCCTGA
GACTGAGCTGTGCCGCCAGCGGCAGACCCGTGTCCAATTACGCCGCTGCCTGGT
TCCGGCAGGCCCCTGGCAAAGAGAGAGAGTTCGTCAGCGCCATCAACTGGCAGA
AAACCGCCACCTACGCCGACAGCGTGAAGGGCCGGTTCACCATCAGCCGGGACA
ACGCCAAGAACAGCCTGTACCTGCAGATGAACTCCCTGCGGGCCGAGGACACCG
CCGTGTACTACTGCGCCGCTGTGTTCCGGGTGGTGGCCCCCAAGACCCAGTACG
ACTACGATTACTGGGGCCAGGGCACCCTGGTCACCGTGTCATCTGGCGGAGGGG
GAGAAGGCGGGGGAGGGGAAGGGGGAGGCGGCGAAGTCCAGCTGCTGGAATCTG
GGGGGGGACTGGTGCAGCCAGGCGGCTCCCTCAGACTGTCTTGCGCCGCCTCCG
GCCGGATCAGCAGCATCATCCACATGGCCTGGTTTAGACAGGCTCCCGGAAAAG
AACGCGAGCTGGTGTCCGAGATCTCCAGAGTGGGCACCACCGTGTATGCCGACT
CCGTGAAAGGCAGATTCACAATCTCCCGCGACAACAGCAAGAATACTCTGTATC
TCCAGATGAATAGCCTGAAGCCCGAAGATACAGCCGTCTACTATTGCAACGCCC
TGCAGTACGAGAAGCACGGCGGAGCCGACTATTGGGGACAGGGAACACTCGTGA
CAGTGTCTAGCTGATGA.

In some embodiments, the fusion protein is encoded by a nucleic acid sequence having at least 90% (e.g., at least 91%, 92%, 93%, 94%, 95%, 96%, 97%, 98%, 99%, or 100%) identity to the nucleic sequence of SEQ ID NO: 57). In some embodiments, the fusion protein is encoded by a nucleic acid sequence of SEQ ID NO: 57.

In some embodiments, the fusion protein has an amino acid sequence having at least 90% identity (e.g., 91%, 92%, 93%, 94%, 95%, 96%, 97%, 98%, 99%, or 100%) to the amino acid sequence:

(SEQ ID NO: 1)
QVQLVESGGG LVKPGGSLRL SCAASGRPVS
NYAAAWFRQA PGKEREFVSA INWQKTATYA
DSVKGRFTIS RDNAKNSLYL QMNSLRAEDT
AVYYCAAVFR VVAPKTQYDY DYWGQGTLVT
VSSGGGGEGGGGEGGGGEVQ LLESGGGLVQ
PGGSLALSCA ASGRISSIIH MAWFRQAPGK
ERELVSEISR VGTTVYADSV KGRFTISRDN
SKNTLYLQMN SLKPEDTAVY YQNALQYEKH
GGADYWGQGT LVTVSS.

In some embodiments, the fusion protein has an amino acid sequence having at least 95% identity (e.g., 95%, 96%, 97%, 98%, 99%, or 100%) to SEQ ID NO: 1. In some embodiments, the fusion protein has an amino acid sequence of SEQ ID NO: 1. In some embodiments, the fusion protein has an amino acid sequence of SEQ ID NO: 1 having a conversion of the N-terminal glutamine of the sequence of SEQ ID NO: 1 to pyro-glutamate.

In some embodiments, the C-terminal residue of the properdin-binding domain of the fusion protein can be fused either directly or via a linker to the N-terminal residue of the human serum albumin binding domain. In other embodiments, the C-terminal residue of the complement component human serum albumin binding domain of the fusion protein can be fused either directly or via a peptide to the N-terminal residue of the properdin binding domain. The fusion proteins described herein may include one or more modified amino acid residues. For example, the amino acid sequence of SEQ ID NO: 1 may include one or more amino acid modifications. The amino acid modifications described herein include all amino acid modifications known in the art (see, e.g., Liu et al., The Journal of Biological Chemistry 286(13:11211-11217, 2011 and Manning et al., Pharmaceutical Research 27(4):544-575, 2010). In all contexts, known conversions of specific amino acids, e.g., during processing or purification of the fusion polypeptide, are to be included, e.g., conversion of an exposed N-terminal glutamine to pyro-glutamate.

### Linkers

As described herein, a linker is used to describe a linkage or connection between polypeptides or protein domains and/or associated non-protein moieties. In some embodiments, a linker is a linkage or connection between at least two polypeptide constructs, e.g., such that the two polypeptide constructs are joined to each other in tandem series (e.g., a monovalent antibody linked to a second polypeptide or monovalent antibody). A linker can attach the N-terminus or C-terminus of one antibody construct to the N-terminus or C-terminus of a second polypeptide construct.

Described herein are fusion proteins that comprise engineered proteins that specifically bind albumin and properdin, wherein the engineered proteins are fused directly or are linked via one or more suitable linkers or spacers. A peptide linker can be, for example, inserted or included at the transition between the engineered proteins of the fusion protein at the sequence level. The identity and sequence of amino acid residues in the linker may vary depending on the desired secondary structure.

A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two antibody constructs, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react through synthetic chemistry means to form a chemical bond, thus connecting the two proteins together. Such chemical conjugation procedures are routine for those skilled in the art.

As described herein, a linker between two peptide constructs can be an amino acid linker including from 1-200 (e.g., 1-4, 1-10, 1-20, 1-30, 1-40, 2-10, 2-12, 2-16, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200) amino acids. Suitable peptide linkers are known in the art, and include, for example, peptide linkers containing flexible amino acid residues such as glycine and serine).

Glycine, serine, and alanine are useful for linkers having maximum flexibility. Any amino acid residue can be considered as a linker in combination with one or more other amino acid residues, which may be the same as or different from the first amino acid residue, to construct larger peptide linkers as necessary depending on the desired properties. In other embodiments, the linker is GGGGEGGGGEGGGGE (SEQ ID NO:10). In other embodiments, the linker is GGGGSGGGGSGGGGS (SEQ ID NO:11). Additional peptide linkers suitable for use in creating fusion proteins described herein include, for example, G₄S (SEQ ID NO:12), (G₄S)₂ (SEQ ID NO:13), (G₄S)₃ (SEQ ID NO:14), (G₄S)₄ (SEQ ID NO:15), (G₄S)₅ (SEQ ID NO:16), (G₄S)₆ (SEQ ID NO:17), (EAAAK)₃ (SEQ ID NO:18), PAPAP (SEQ ID NO:19), G₄SPAPAP (SEQ ID NO:20), PAPAPG₄S (SEQ ID NO:21), (GGGDS)₂ (SEQ ID NO:22), (GGGES)₂ (SEQ ID NO:23), GGGDSGGGGS (SEQ ID NO:24), GGGASGGGGS (SEQ ID NO:25), GGGESGGGGS (SEQ ID NO:26), ASTKGP (SEQ ID NO:27), ASTKGPSVFPLAP (SEQ ID NO:28), G₃P (SEQ ID NO:29), G₇P (SEQ ID NO:30), PAPNLLGGP (SEQ ID NO:31), G₆ (SEQ ID NO:32), G₁₂ (SEQ ID NO:33), APELPGGP (SEQ ID NO:34), SEPQPQPG (SEQ ID NO:35), (G₃S₂)₃ (SEQ ID NO:36), GGGGGGGGGSGGGS (SEQ ID NO:37), GGGGSGGGGGGGGGS (SEQ ID NO:38), (GGSSS)₃ (SEQ ID NO:39), (GS₄)₃ (SEQ ID NO:40), G₄A(G₄S)₂ (SEQ ID NO:41), G₄SG₄AG₄S (SEQ ID NO:42), G₃AS(G₄S)₂ (SEQ ID NO:43), G₄SG₃ASG₄S (SEQ ID NO:44), G₄SAG₃SG₄S (SEQ ID NO:45), (G₄S)₂AG₃S (SEQ ID NO:46), G₄SAG₃SAG₃S (SEQ ID NO:47), G₄D(G₄S)₂ (SEQ ID NO:48), G₄SG₄DG₄S (SEQ ID NO:49), (G₄D)₂G₄S (SEQ ID NO:50), G₄E(G₄S)₂ (SEQ ID NO:51), G₄SG₄EG₄S (SEQ ID NO:52), (G₄E)₂G₄S (SEQ ID NO:53), and GGGGAGGGGAGGGGS (SEQ ID NO: 54). One of skill in the art can select a linker, for example, to reduce or eliminate post-translational modification, e.g., glycosylation, e.g., xylosylation. In certain embodiments, the fusion protein comprises at least two sdAbs, Dabs, VHH antibodies, VHH antibody fragments, or combination thereof wherein at least one of the sdAbs, Dabs, VHH antibodies, or VHH antibody fragments is directed against albumin and one of the sdAbs, Dabs, VHH antibodies, or VHH antibody fragments is directed against properdin, so that the resulting fusion protein is multivalent or multi-specific. The binding domains or moieties can be directed against, for example, HSA, cynomolgus monkey serum albumin, human properdin and/or cynomolgus monkey properdin.

### Methods of Formulating a High Concentration Composition

Described herein are methods of developing a high concentration formulation of a fusion protein for subcutaneous administration. Developing high concentration formulations for subcutaneous administration has several challenges including the need to optimize viscosity, stability, and delivery. A Design of Experiment (DOE) approach may be used to develop a high concentration formulation for subcutaneous administration of a fusion protein to a subject. The methods described here are designed to select potential viscosity reducing excipients while having minimal negative impact on stability of the pharmaceutical compositions.

Formulating a high concentration formulation of fusion protein may include a first step of performing an excipient screening. The excipient screening may include measuring the stability and viscosity of a solution including the fusion protein and one or more excipients over a period of time. In the second step, the method may include a measuring viscosity, turbidity, and formation rate of high molecular weight compounds in the solution, following the excipient screening, as a result of changing pH, acetate concentration, and sucrose concentration. As a third step, the method may include measuring viscosity, turbidity, and formation rate of high molecular weight compounds in the solution as a result of changing pH, sucrose concentration, excipient, and concentration of the fusion protein. A viscosity assessment of the solution may be performed as the fourth step. The viscosity of the formulation is measured at a range of temperatures and a range of fusion protein concentrations.

The measuring of viscosity, turbidity, and formation rate of high molecular weight compounds in the second and third steps may be performed weekly and may be measured is for at least 4 weeks.

For the excipient screening, the one or more excipients may be selected from succinate, arginine, acetate, or a combination thereof. The one or more excipients may be arginine. For example, the arginine is arginine hydrochloride, arginine acetate, or arginine succinate. The one or more excipients may be succinate. In some embodiments, the one or more excipients is succinate and arginine hydrochloride. The one or more excipients may be acetate. In some embodiments, the one or more excipients is acetate and arginine hydrochloride. In some embodiments, the one or more excipients is acetate and arginine acetate.

In some embodiments, the solution described in step two has a concentration of acetate of between 10 mM and 200 mM (e.g., between 10 mM and 150 mM, 10 mM and 100 mM, 10 mM and 50 mM, 50 mM and 200 mM, 100 mM and 200 mM, or 150 mM and 200 mM). For example, the solution has a concentration of acetate of between 15 mM and 100 mM (e.g., between 15 mM and 80 mM, 15 mM and 60 mM, 15 mM and 40 mM, 15 mM and 20 mM, 20 mM and 100 mM, 40 mM and 100 mM, 60 mM and 100 mM, or 80 mM and 100 mM).

The solution in the first step including the excipient screening may also sucrose. The sucrose may have a concentration in the solution of between 1% (w/v) and 10% (w/v) (e.g., 1% (w/v), 2% (w/v), 3% (w/v), 4% (w/v), 5% (w/v), 6% (w/v), 7% (w/v), 8% (w/v), 9% (w/v), or 10% (w/v)). In some embodiments, the sucrose has a concentration of about 4% (w/v). The solution in the second step may have a concentration of sucrose of between 1% (w/v) and 20% (w/v) (e.g., between 1% (w/v) and 15% (w/v), 1% (w/v) and 10% (w/v), 1% (w/v) and 5% (w/v), 5% (w/v) and 20% (w/v), 10% (w/v) and 20% (w/v), or 15% (w/v) and 20% (w/v)). For example, the solution described in step two may have a concentration of sucrose of between 2% (w/v) and 10% (w/v) (e.g., 2% (w/v), 3% (w/v), 4% (w/v), 5% (w/v), 6% (w/v), 7% (w/v), 8% (w/v), 9% (w/v), or 10% (w/v)). The third step may have a solution with a concentration of sucrose of between 0% (w/v) and 10% (w/v) (e.g., 1% (w/v), 2% (w/v), 3% (w/v), 4% (w/v), 5% (w/v), 6% (w/v), 7% (w/v), 8% (w/v), 9% (w/v), or 10% (w/v)). For example, the solution in step three may have a concentration of sucrose of between 0% (w/v) and 5% (w/v) (e.g., 1% (w/v), 2% (w/v), 3% (w/v), 4% (w/v), or 5% (w/v)).

The pH of the solution in the second step may be between 4 and 7 (e.g., a pH between 4 and 6, 4 and 5, 5 and 7, or 6 and 7). For example, the solution has a pH of between 4.5 and 6.0 (e.g., a pH of between 4.5 and 5.5, 4.5 and 5, 5 and 6, or 5.5 and 6). The pH of the solution in the third step may be between 5 and 6.

The fusion protein present in the high concentration formulation may include 6 CDRs having amino acid sequences of SEQ ID NOS: 2-7. The fusion protein may include a first portion that comprises an amino acid sequence having at least 95% (e.g., at least 96%, 97%, 98%, 99%, or 100%) sequence identity to SEQ ID NO: 55. Furthermore, the fusion protein may include a second portion including an amino acid sequence having at least 95% (e.g., at least 96%, 97%, 98%, 99%, or 100%) sequence identity SEQ ID NO: 56. In some embodiments, the fusion protein has an amino acid sequence of SEQ ID NO: 1, or a modification thereof. In some embodiments, the modification comprises conversion of the N-terminal glutamine of the sequence of SEQ ID NO: 1 to pyro-glutamate.

The fusion protein may have a concentration in the solution described in the second step of about 150 mg/mL. The fusion protein may have a concentration in the solution described in the second step of about 190 mg/mL. The solution in the second step may have a fusion protein concentration of between about 150 mg/mL and 230 mg/mL (e.g., 150 mg/mL and 200 mg/mL, 150 mg/mL and 175 mg/mL, 175 mg/mL and 230 mg/mL, 200 mg/mL and 230 mg/mL, or 220 mg/mL and 230 mg/mL). In some embodiments, the solution of in the third step has a fusion protein concentration of between about 100 mg/mL and 300 mg/mL (e.g., between 100 mg/mL and 250 mg/mL, 100 mg/mL and 200 mg/mL, 100 mg/mL and 150 mg/mL, 150 mg/mL and 300 mg/mL, 200 mg/mL and 300 mg/mL, or 250 mg/mL and 300 mg/mL). The fusion protein in the high concentration formulation may be present in a concentration of at least 150 mg/mL. For example, the fusion protein in the excipient screening may be present at a concentration of about 200 mg/mL.

The high concentration formulation may have a viscosity of between 5 cP and 15 cP (e.g., 5 cP, 6 cP, 7 cP, 8 cP, 9 cP, 10 cP, 11 cP, 12 cP, 13 cP, 14 cP, or 15 cP) at 20° C. For example, the formulation has a viscosity of about 10 cP or 11 cP at 20° C. The formulation may have a percentage of higher molecular weight compounds per month at 25° C. over one month of between 0.05% (w/v) and 0.5% (w/v) (e.g., between 0.05% (w/v) and 0.4% (w/v), 0.05% and 0.3% (w/v), 0.05% and 0.2% (w/v), 0.05% (w/v) and 0.1% (w/v), 0.1% (w/v) and 0.5% (w/v), 0.2% (w/v) and 0.5% (w/v), 0.3% (w/v) and 0.5% (w/v), or 0.4% (w/v) and 0.5% (w/v)). For example, the formulation has a percentage of higher molecular weight compounds per month at 25° C. over one month of about 0.2% (w/v). In some embodiments, the formulation has a percentage of higher molecular weight compounds per month at 37° C. over one month of between about 1% (w/v) and 10% (w/v) (e.g., 1% (w/v), 2% (w/v), 3% (w/v), 4% (w/v), 5% (w/v), 6% (w/v), 7% (w/v), 8% (w/v), 9% (w/v), or 10% (w/v)). In some embodiments, the formulation has a percentage of higher molecular weight compounds per month at 37° C. over one month of about 5% (w/v). In some embodiments, the formulation has a turbidity of between about 1 and 10 (e.g., 1, 2, 3, 4, 5, 6, 7, 8, 9, or 10) per month at 37° C., wherein turbidity is measured as the optical density at 400 nm. In some embodiments, the formulation has a turbidity of about 3 or 4 per month at 37° C.

### Methods of Treatment

The compositions described herein can be used in methods of treating a disease or disorder mediated by alternative complement pathway dysfunction in an individual in need of such treatment, the method including administering to the individual a therapeutically effective amount of a high concentration formulation that includes fusion protein described herein in treating diseases mediated by alternative complement pathway dysregulation by inhibiting the alternative complement pathway activation in a mammal (e.g., a human). In some embodiments, the pharmaceutical composition described herein may be used in a method of treating or prevention a disease or condition in a subject, wherein the disease is sickle cell disease.

The pharmaceutical compositions described herein can be administered by a variety of methods known in the art, although for many therapeutic applications, the preferred route/mode of administration is intravenous injection or infusion. The polypeptide can also be administered by intramuscular or subcutaneous injection. In some embodiments, the pharmaceutical compositions described herein may be administered to a subject subcutaneously. As will be appreciated by the skilled artisan, the route and/or mode of administration will vary depending upon the desired results. Many methods for the preparation of such formulations are known to those skilled in the art (e.g., Sustained and Controlled Release Drug Delivery Systems, J. R. Robinson, ed., Marcel Dekker, Inc., New York, 1978). Methods applicable to the controlled or extended release of antibodies such as the monovalent single domain antibodies disclosed herein are known (U.S. Pat. Nos. 6,306,406 and 6,346,274; U.S. Patent Application Nos: US20020182254 and US20020051808, the entire teachings of each of which are incorporated herein by reference).

In some embodiments, the fusion proteins described herein are administered using a pre-filled syringe. In other embodiments, the fusion protein is administered using an autoinjector device. For example, the autoinjector device may include a single vial system, such as a pen injector device for solution delivery. Such devices are commercially available from manufacturers such as BD Pens, BD Autojector®, Humaject®, NovoPen®, B-D® Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, DosePro®, Medi-Ject®, e.g., as made or developed by Becton Dickinson (Franklin Lakes, NJ), Ypsomed (Burgdorf, Switzerland, www.ypsomed.com; Bioject, Portland, OR.; National Medical Products, Weston Medical (Peterborough, UK), Medi-Ject Corp (Minneapolis, MN), and Zogenix, Inc, Emeryville, CA. Recognized devices comprising a dual vial system include those pen-injector systems for reconstituting a lyophilized drug in a cartridge for delivery of the reconstituted solution such as the HumatroPen®. In one embodiment, the autoinjector is a YpsoMate 2.25 or YpsoMate 2.25 Pro (Ypsomed) disposable injection device.

### Kits

A composition containing the fusion protein described herein can be provided in a kit for use in of a disease or condition. The kit can further include a label or package insert that instructs a user of the kit, such as a subject having a disease or condition or a physician, to perform the methods described herein. In some embodiments, the kit includes a container having a label and a composition including the fusion protein described herein and the label indicates that the composition is to be administered to a patient in need thereof. The kit may optionally include a syringe or other device (e.g., autoinjector, pre-filled syringe, or wearable device) for administering the composition. In other embodiments, the kit includes single or multi-chambered pre-filled syringes (e.g., liquid syringes and lyosyringes). In some embodiments, the kit includes cartridges containing a composition described herein for use with a medical device (e.g., an autoinjector or a wearable device).

### EXAMPLES

The following examples are put forth so as to provide those of ordinary skill in the art with a description of how the compositions and methods described herein may be used, made, and evaluated, and are intended to be purely exemplary and are not intended to limit the scope of the present disclosure.

### Example 1. Application of Design Experiment Strategy to Develop a Bispecific Antibody High Concentration Formulation for Subcutaneous Administration

Subcutaneous (SC) administration of protein therapeutics offers several advantages including improved patient compliance/convenience, preference, and PK/PD profile. The injection volume for SC administration is typically small (<2 mL), which in turn leads to a need for high concentration formulation to achieve the appropriate dose. Several challenges remain associated with developing a high concentration formulation such as viscosity, stability, and delivery.

A Design of Experiment (DOE) approach was used to develop a high concentration formulation for subcutaneous administration of a 27 kDa bispecific antibody to a subject. Given that viscosity can be a limiting factor for the development of ultra-high concentration (e.g., >150 mg/mL) formulations, the goal of the experiment was to select potential viscosity reducing excipients while having minimal negative impact on stability of the pharmaceutical compositions.

A preliminary excipient screen with arginine, arginine and counter ions, and proline was performed to assess the stability of a solution having a concentration of 200 mg/mL of a fusion protein having the amino acid sequence of SEQ ID NO: 1. After the preliminary excipient screen, a 1^{st} DOE was performed to assess the impact of pH, acetate concentration, and sucrose concentration had on the stability of the pharmaceutical composition having a fusion protein concentration of 150 mg/mL. Following this assessment, the impact of pH, protein concentration, presence of proline, and sucrose concentration were studied with respect to the stability of the pharmaceutical composition. Finally, a viscosity assessment of the pharmaceutical compositions was performed.

For the preliminary excipient screen the following formulations were studied:
F1: 20 mM acetate, 8.6% w/v sucrose, 0.05% (w/v) PS-80, pH 5.4
F2: 20 mM acetate, 4% (w/v) sucrose, 0.05% (w/v) PS-80, pH 5.4
F3: 20 mM acetate, 4% (w/v) sucrose, 150 mM Arg-HCl, 0.05% (w/v) PS-80, pH 5.4
F4: 20 mM succinate, 4% (w/v) sucrose, 150 mM Arg-HCl, 0.05% (w/v) PS-80, pH 5.4
F5: 20 mM acetate, 4% (w/v) sucrose, 150 mM Arg-acetate, 0.05% (w/v) PS-80, pH 5.4
F6: 4% (w/v) sucrose, 150 mM Arg-succinate, 0.05% (w/v) PS-80, pH 5.4
F7: 20 mM acetate, 4% (w/v) sucrose, 150 mM proline, 0.05% (w/v) PS-80, pH 5.4 (1 timepoint post 1 week)

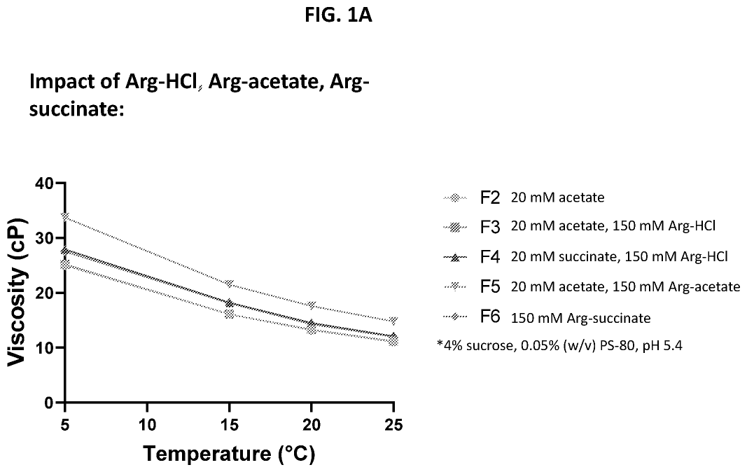

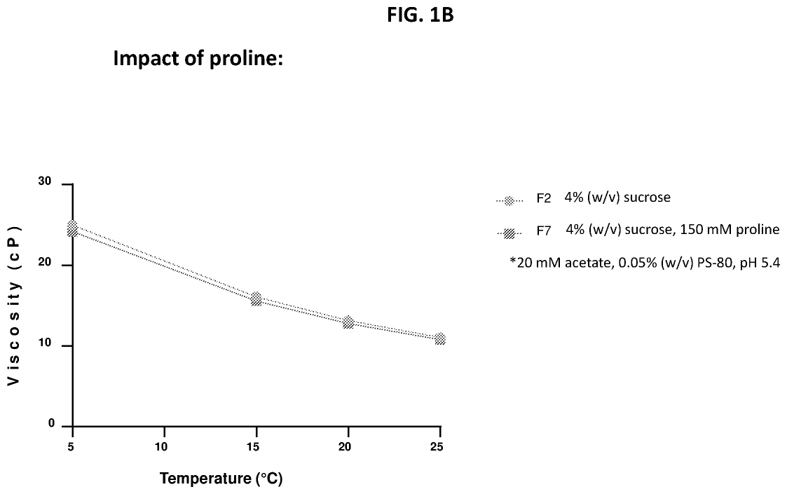

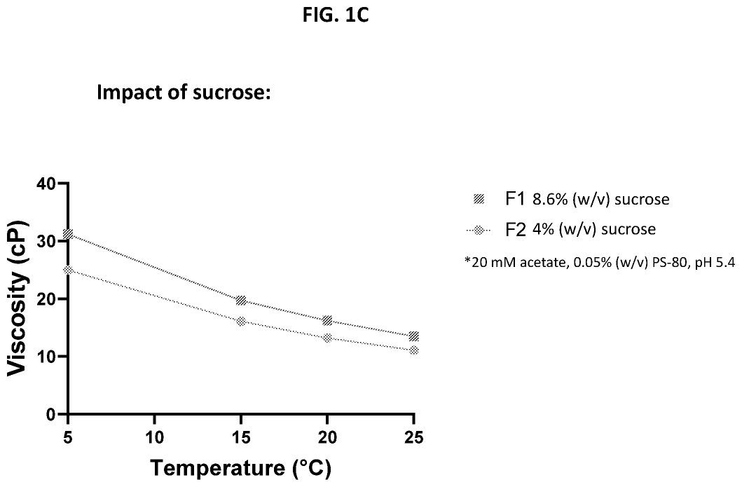

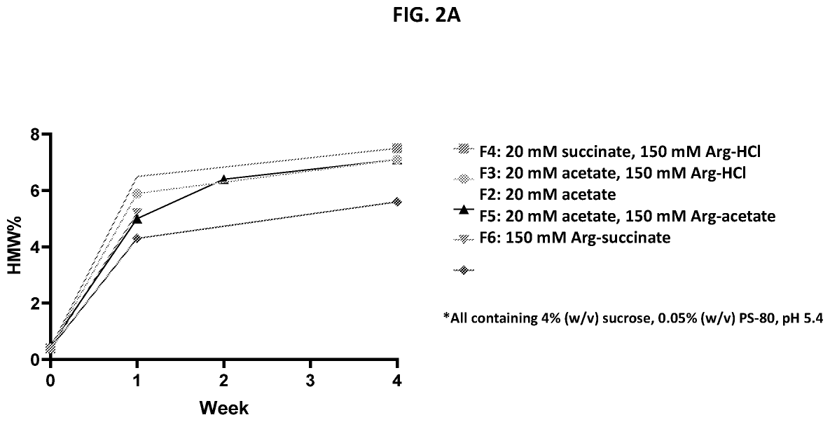

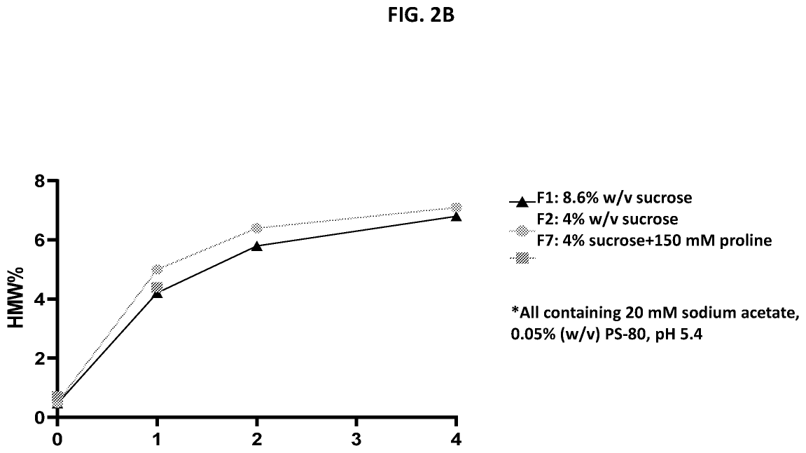

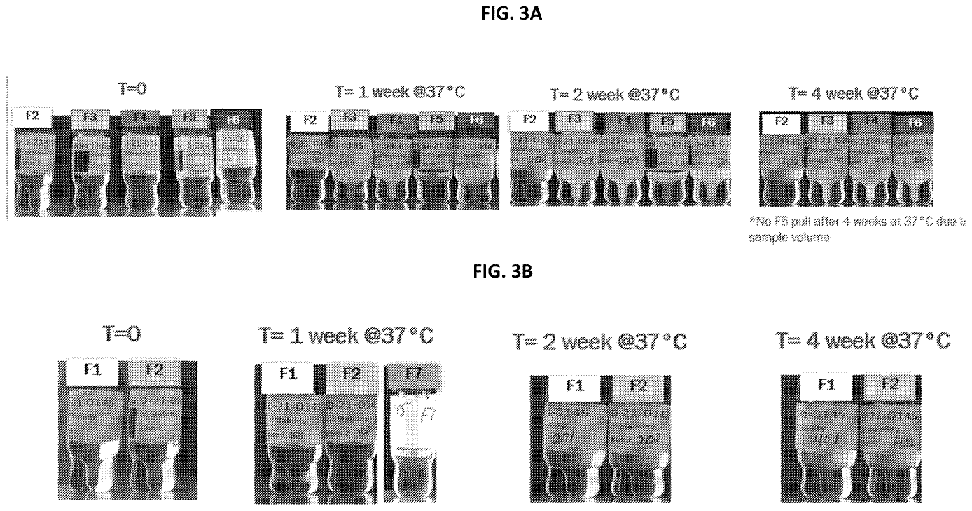

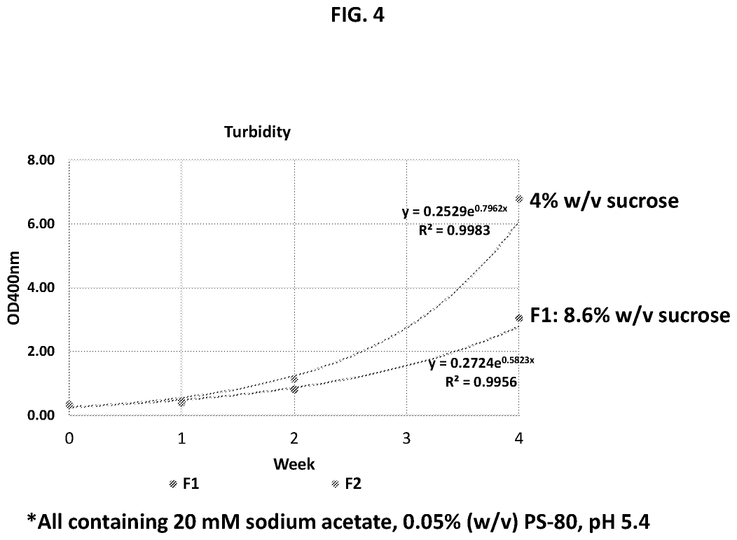

The viscosity (FIG. 1A, FIG. 1B, and FIG. 1C), percentage of higher molecular weight (HMW) species present in solution (FIG. 2A and FIG. 2B), and turbidity of the solution, as determined by the optical density at 400 nm, (FIG. 4) were assessed after storage at 1, 2 and 4 weeks at 37° C. and photographs of the solutions over time were take (FIG. 3A and FIG. 3B). This excipient screen showed that arginine and proline did not reduce viscosity and showed that an increased level of sucrose slightly increases viscosity. Additionally, Arg-HCl was shown to destabilize the molecule with increasing turbidity and HMW species. Arg-acetate destabilized the molecule with increasing turbidity. Arg-succinate destabilized the molecule with increasing turbidity but lower HMW species. The presence of proline reduced formation of HMW species. Increased concentration of sucrose minimized the increase of turbidity and reduced formation of HMW at 37° C. The formulations with Arg revealed significant increase in turbidity after 1 week at 37° C.

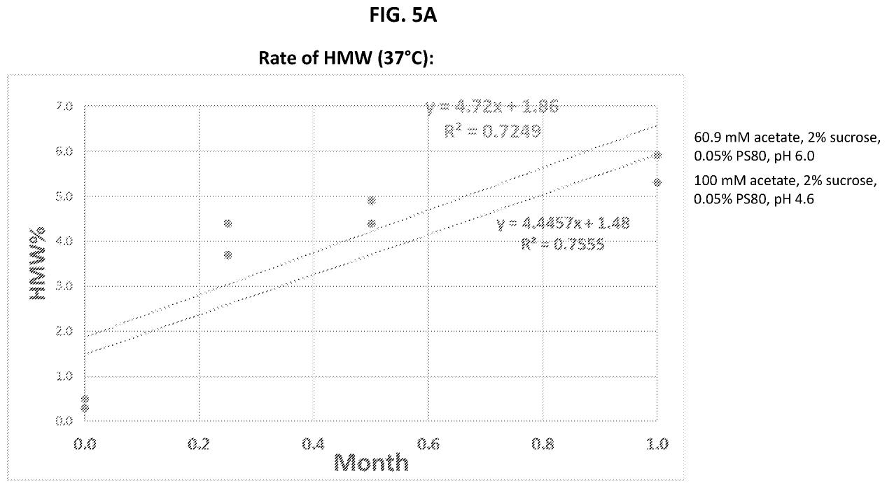

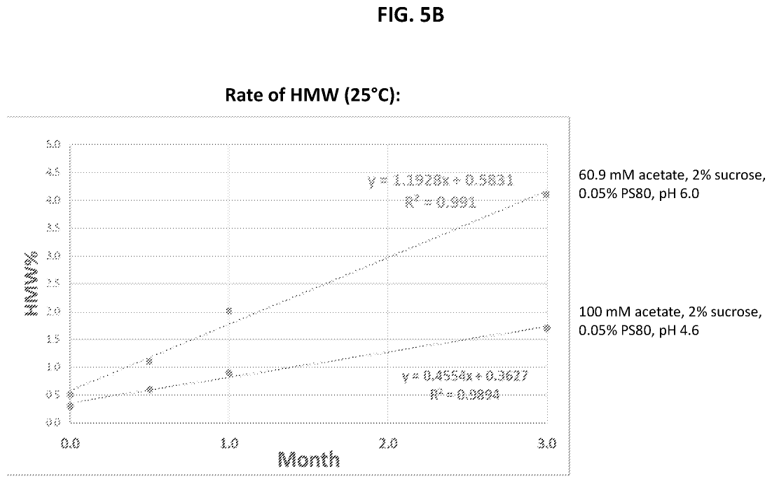

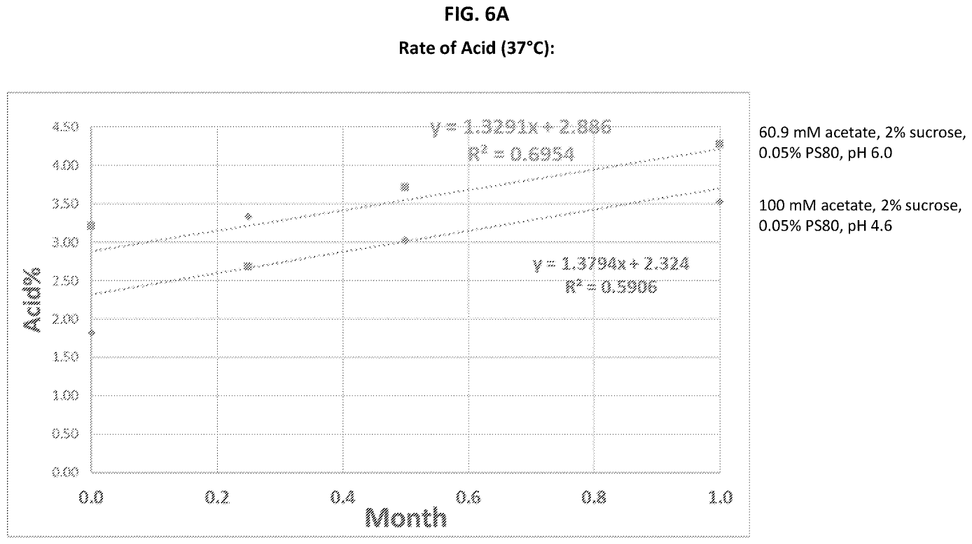

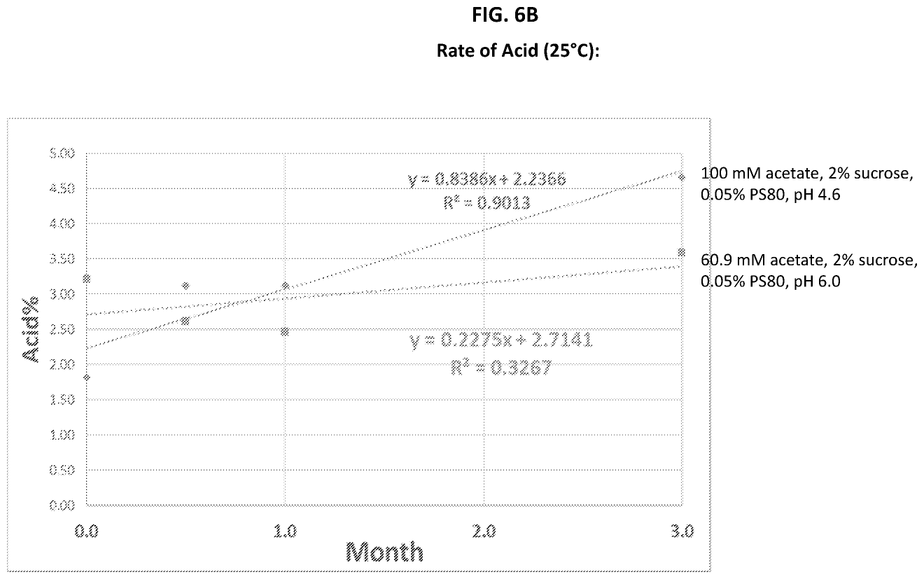

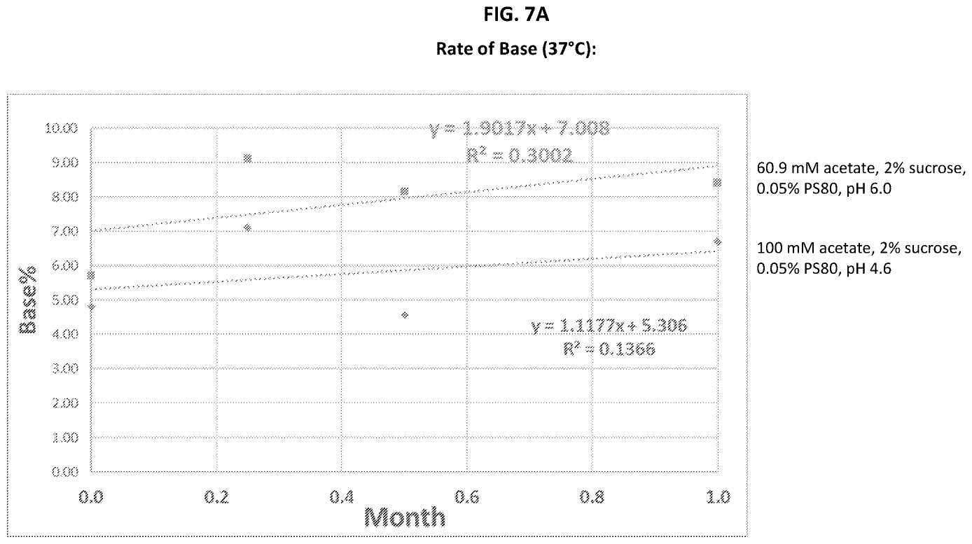

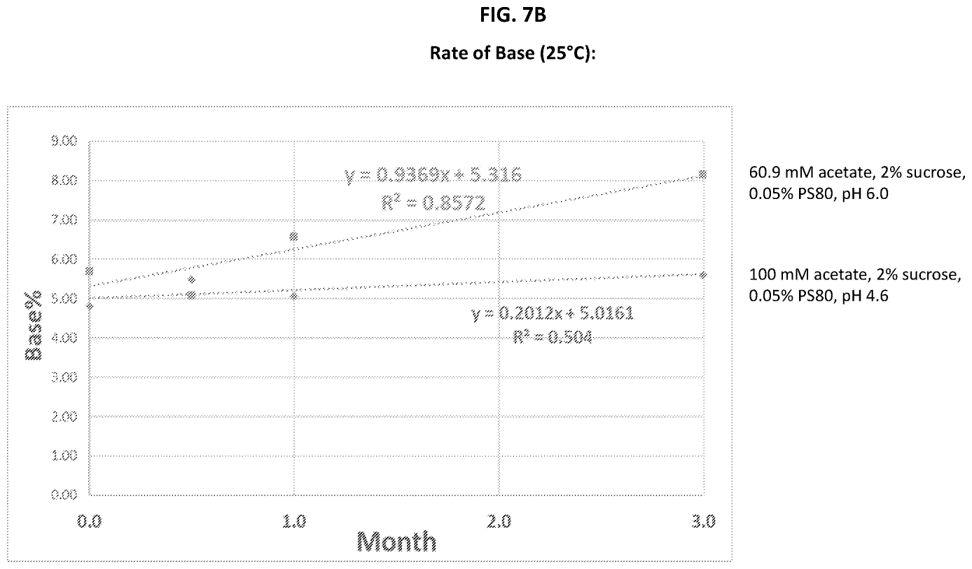

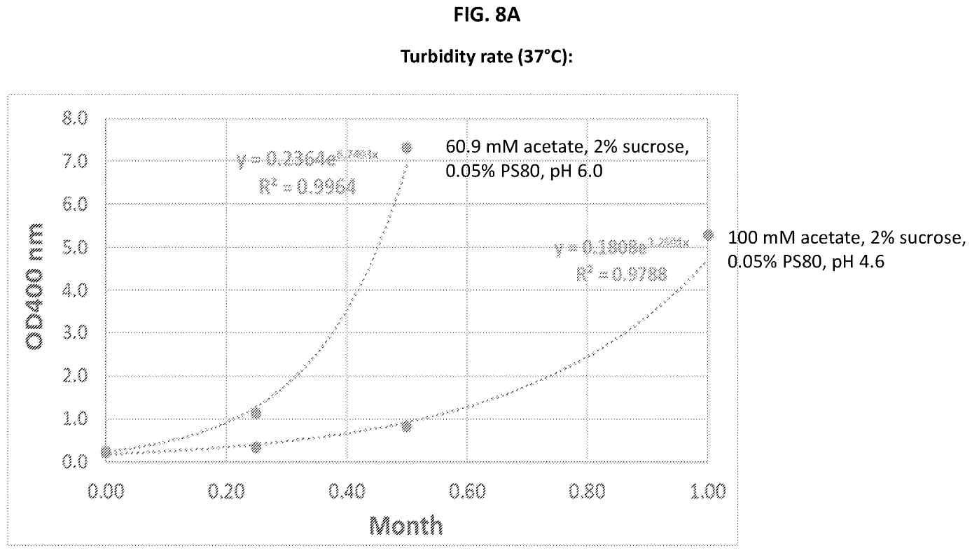

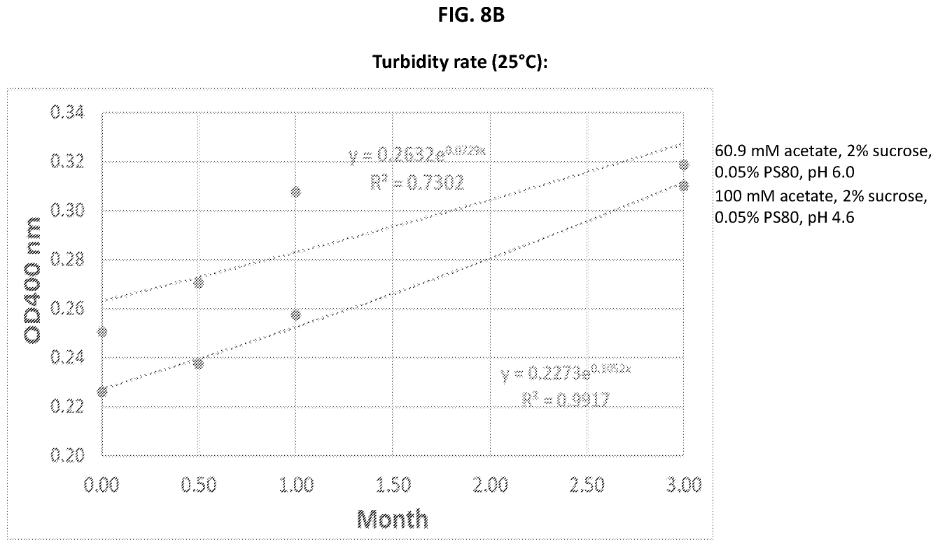

Following the preliminary excipient screen, the 1^{st} DOE was performed to assess the impact of pH, acetate concentration, and sucrose concentration had on the stability of the pharmaceutical composition having a fusion protein concentration of 150 mg/mL. For a composition including 100 mM acetate, 2% (w/v) sucrose, and 0.05% PS80 at pH 5.4 (F4) and a composition including 60.9 mM acetate, 2% (w/v) sucrose, and 0.05% PS80 at pH 6.0 (F5) the rate of HMW formation was assessed over the course of a month at 37° C. (FIG. 5A) and 25° C. (FIG. 5B). The rate of acid formation, rate of base formation, and rate of turbidity increase were all measured as well over the course of a month at both 37° C., as shown in FIGS. 6A, 7A and 8A respectively, and 25° C., as shown in FIGS. 6B, 7B and 8B respectively.

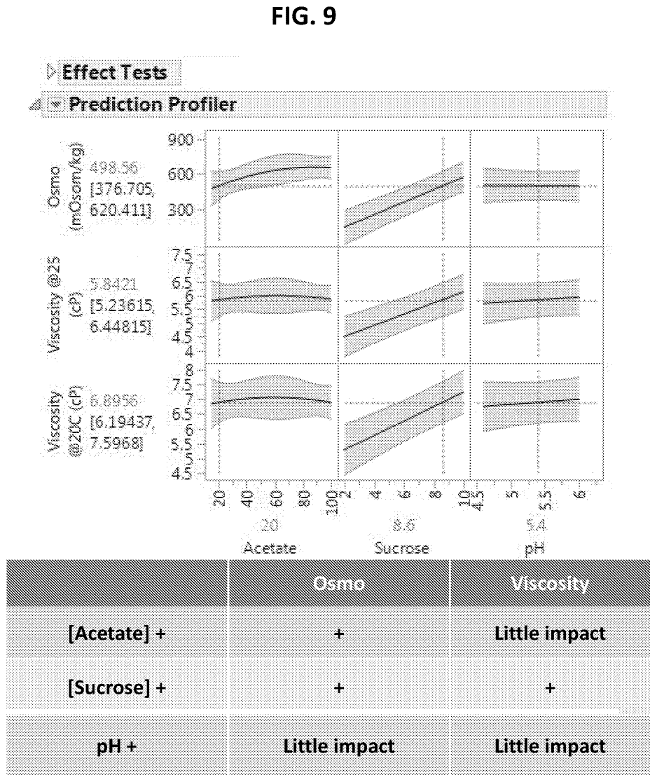

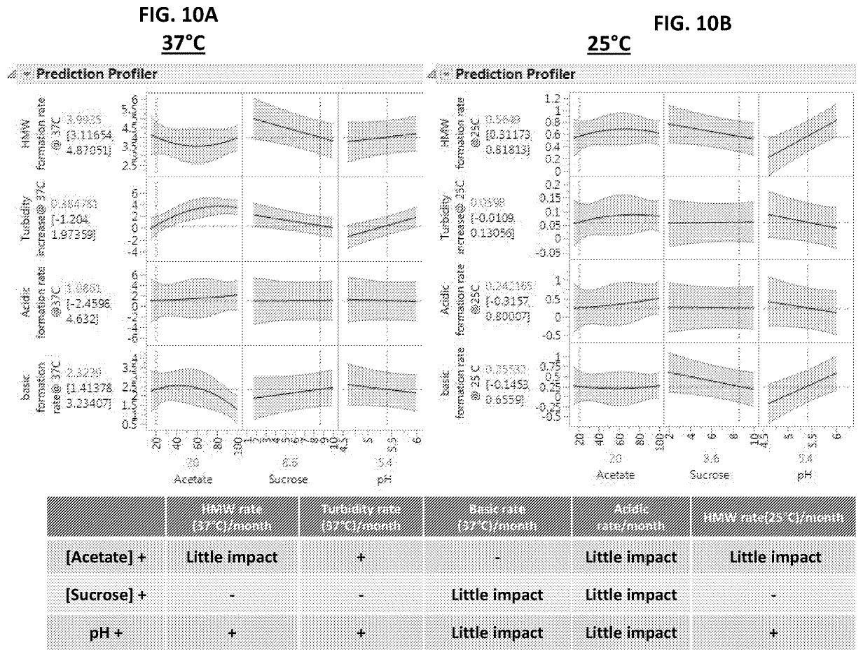

Additionally, prediction profiles were generated for compositions having 150 mg/mL of the fusion protein to study the impact of concentration of acetate, the concentration of sucrose, and the pH of the composition has on osmolality, viscosity at 25° C. and 20° C. (FIG. 9). These profiles showed that an increase in acetate concentration increased osmolality but had little impact on viscosity, and increase in sucrose concentration increased osmolality and increased viscosity, and an increase in pH had little impact on the osmolality and viscosity. Prediction profiles were also generated for compositions to assess the impact concentration of acetate, the concentration of sucrose, and the pH of the composition has on HMW formation rate, turbidity increase, acid formation rate, and base formation rate at 37° C. (FIG. 10A) and 25° C. (FIG. 101B). These predictions showed an increase in acetate concentration had little impact on the rate of HMW formation, resulted in an increase in the turbidity rate, and had little impact on the acidic rate formation. An increase in sucrose concentration had little impact on the basic rate formation and the acidic rate formation. An increase in pH had little impact on the basic rate formation and the acidic rate formation and resulted in an increase in the HMW rate of formation and the turbidity rate. The results of these studies are summarized below in Table 1.

TABLE 1
Summary of results for first DOE

| | Predicated | | Experimental |
|---|---|---|---|
| Osmo | 499 | (377-620) | 402 |
| Viscosity@25° C. | 5.8 | (5.2-6.4) | 5.6 |
| Viscosity@20° C. | 6.9 | (6.2-7.6) | 6.5 |
| HMW %/month@37° C. | 4.0 | (3.1-4.9) | 4.8 |
| HMW %/month @ 25° C. | 0.6 | (0.3-0.8) | 0.3 |
| Acidic/month@37° C. | 1.1 | (−2.5-4.6) | 0.4 |
| Acidic/month @25° C. | 0.2 | (−0.3-0.8) | −0.0 |
| Basic/month@37° C. | 2.3 | (1.4-3.2) | 1.7 |
| Basic/month @25° C. | 0.3 | (−0.1-0.7) | −0.2 |
| Turbidity/month@37° C. | 0.4 | (−1.2-2.0) | 1.3 |

The results of this study resulted in a Phase 1 formulation of 150 mg/mL a bispecific antibody having the sequence of SEQ ID NO:1 in 20 mM sodium acetate, 8.6% w/v sucrose, 0.05% w/v PS80, pH 5.4. Additionally, it was noted that the predicted values aligned with experimental data, indicating the model was robust.

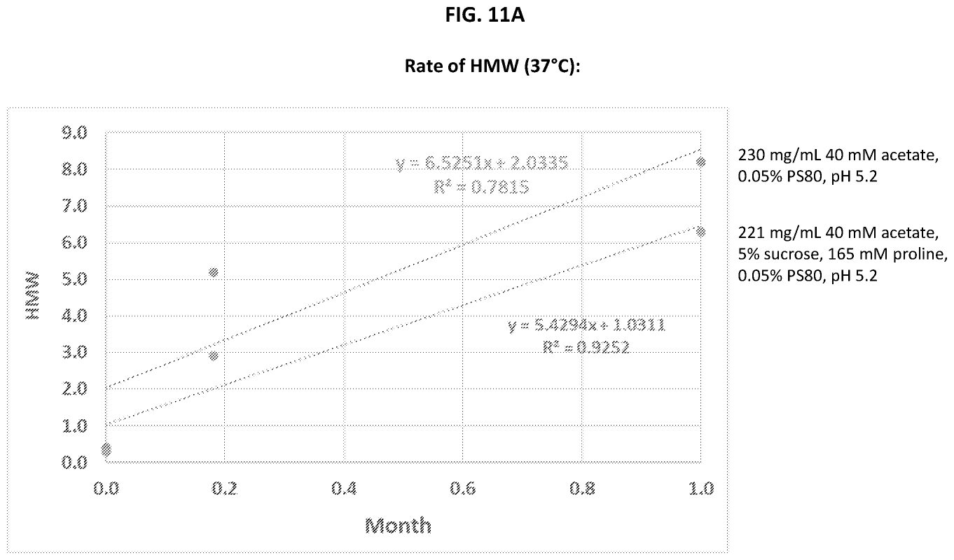

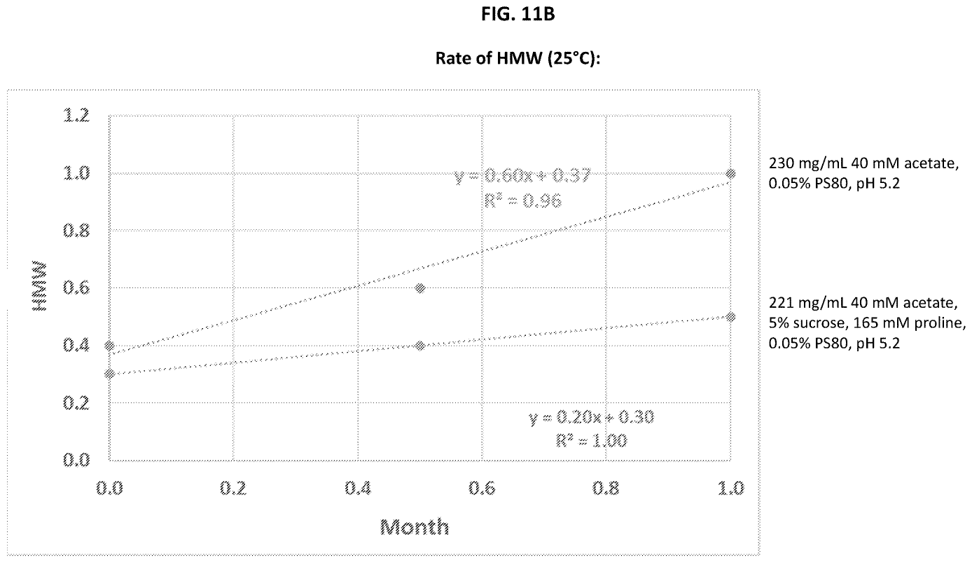

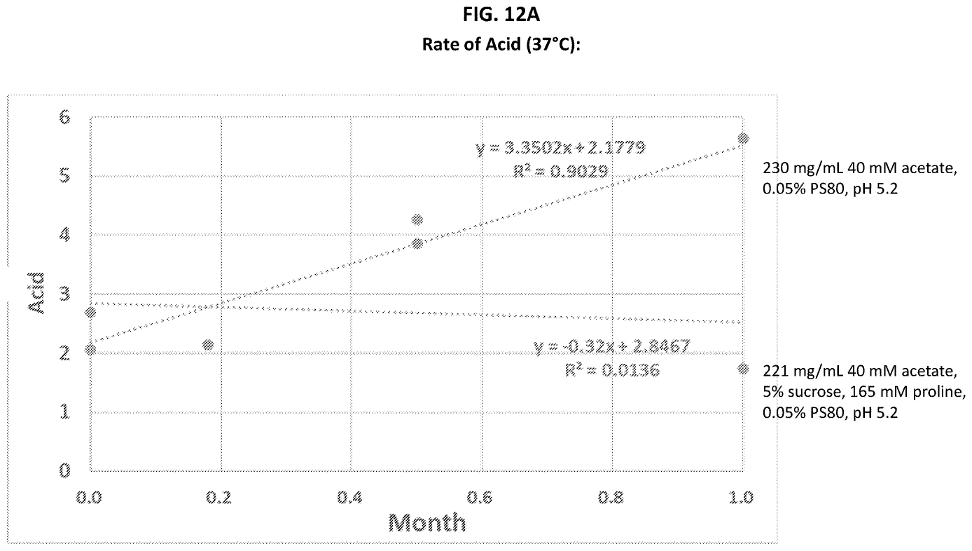

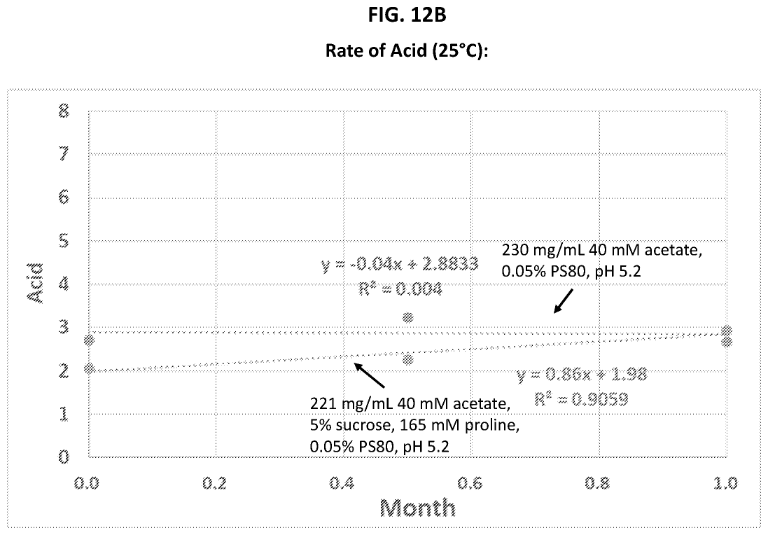

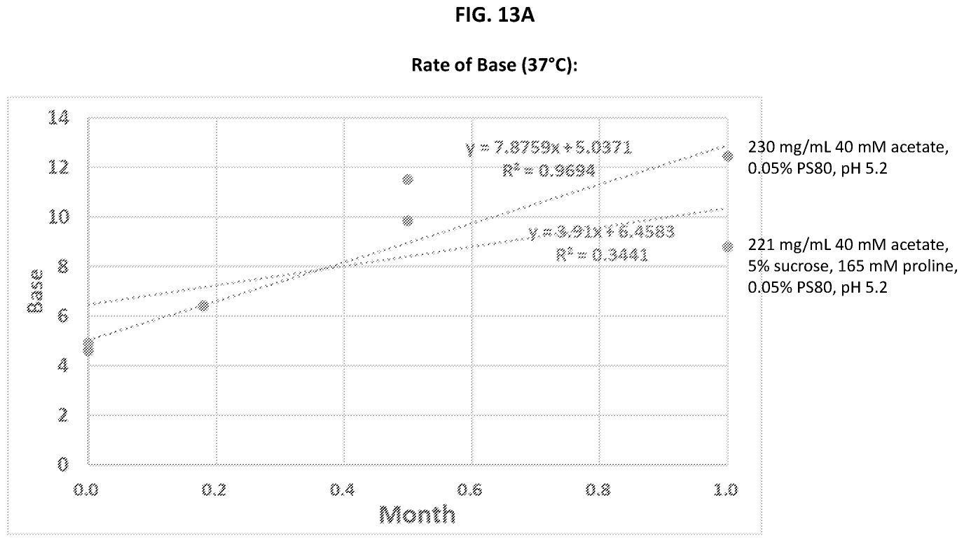

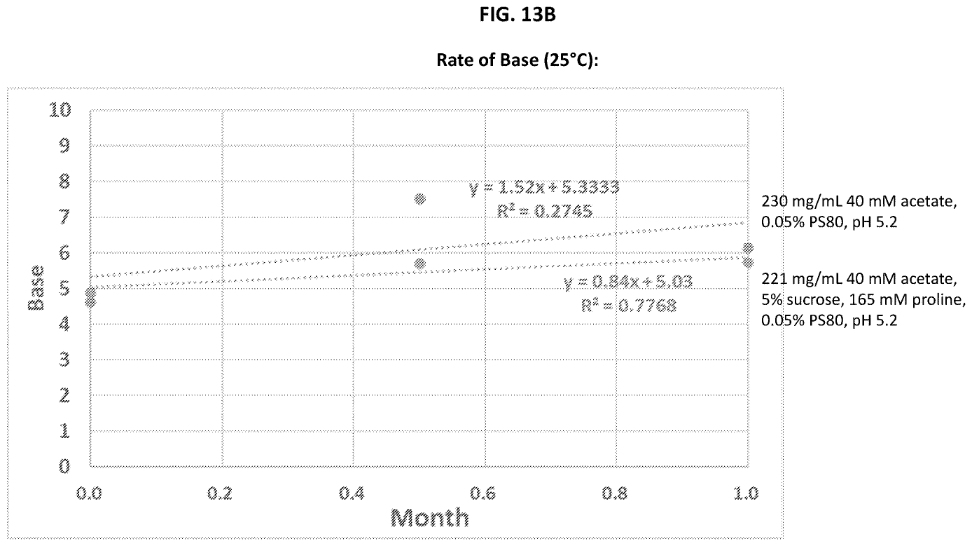

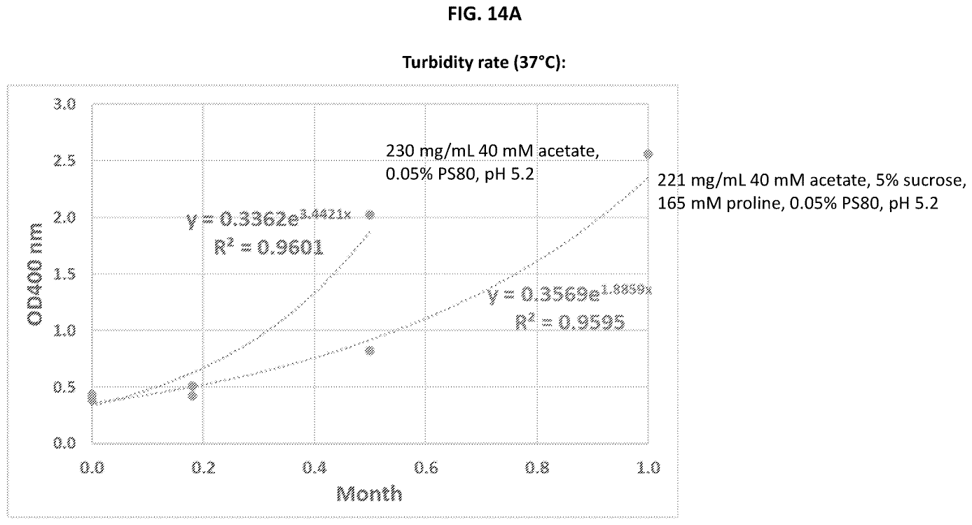

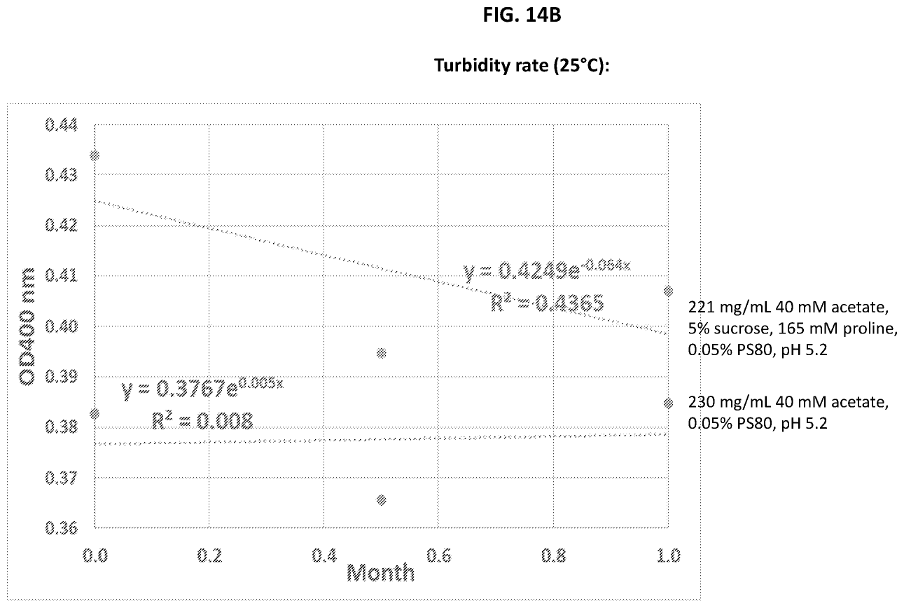

Following, the 1^{st} DOE, the 2^{nd} DOE was performed to assess the impact the impact of pH, protein concentration, presence of proline, and sucrose concentration were studied with respect to the stability of the pharmaceutical composition. For a composition including 230 mg/mL fusion protein, 40 mM acetate, and 0.05% PS80 at pH 5.2 (F2) and a composition including 221 mg/mL, 40 mM acetate, 5% (w/v) sucrose, 165 mM proline, and 0.05% PS80 at pH 5.2 (F7) the rate of HMW formation was assessed over the course of a month at 37° C. (FIG. 11A) and 25° C. (FIG. 11B). The rate of acid formation, rate of base formation, and rate of turbidity increase were all measured as well over the course of a month at both 37° C., as shown in FIGS. 12A, 13A and 14A respectively, and 25° C., as shown in FIGS. 12B, 13B and 14B respectively.

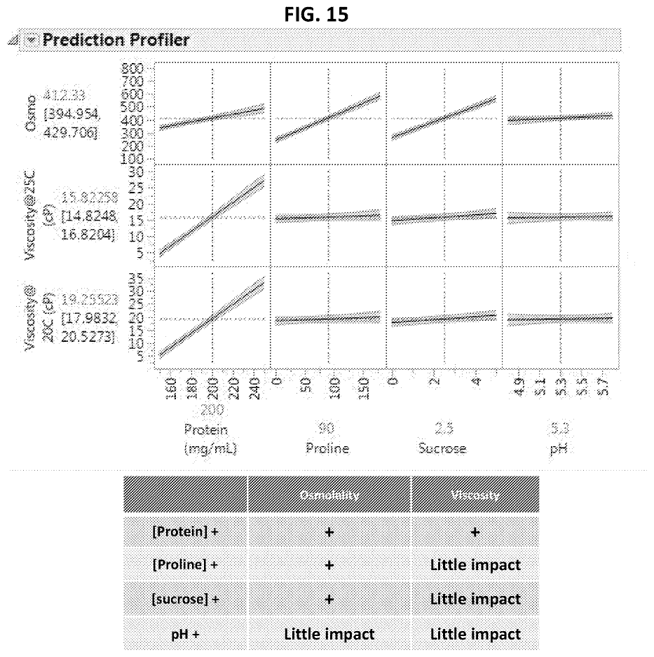

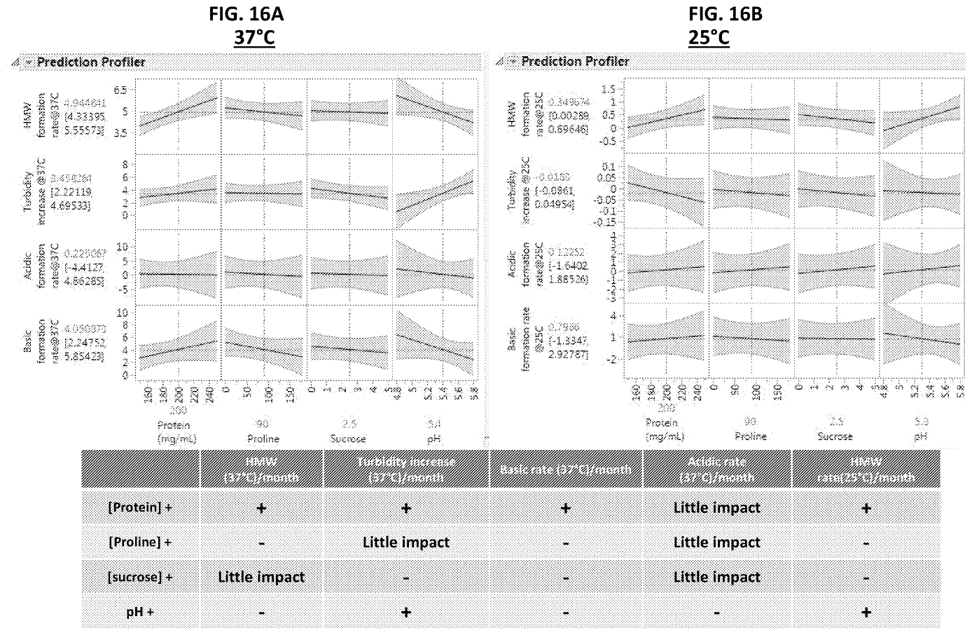

Additionally, prediction profiles were generated for compositions to study the impact of concentration of protein concentration, proline concentration, concentration of sucrose, and the pH of the composition has on osmolality, viscosity at 25° C. and 20° C. (FIG. 15). These profiles showed that an increase in protein concentration increased osmolality and viscosity, an increase in proline concentration increased osmolality and had little impact on viscosity, an increase in sucrose concentration increased osmolality and had little impact to viscosity, and an increase in pH had little impact on the osmolality and viscosity. Prediction profiles were also generated for compositions to assess the impact concentration of protein concentration, proline concentration, sucrose concentration, and the pH of the composition has on HMW formation rate, turbidity increase, acid formation rate, and base formation rate at 37° C. (FIG. 16A) and 25° C. (FIG. 16B). These predictions showed an increase in protein concentration increase the rate of HMW formation, the rate of turbidity, the basic rate formation, and had little impact on the acidic rate formation. An increase in proline concentration resulted in a decrease in HMW species formation and a decrease in basic rate formation and had little impact on the rate of turbidity and the rate of acidic formation. An increase in sucrose concentration had little impact rate of HMW formation at 37° C. and resulted in a decrease in the basic rate formation, the acidic rate formation and HMW formation at 25° C. An increase in pH resulted in a decrease of HMW rate formation at 37° C., basic rate formation, and acidic rate formation and an increase in the rate of turbidity and the rate of HMW formation at 25° C. and the turbidity rate.

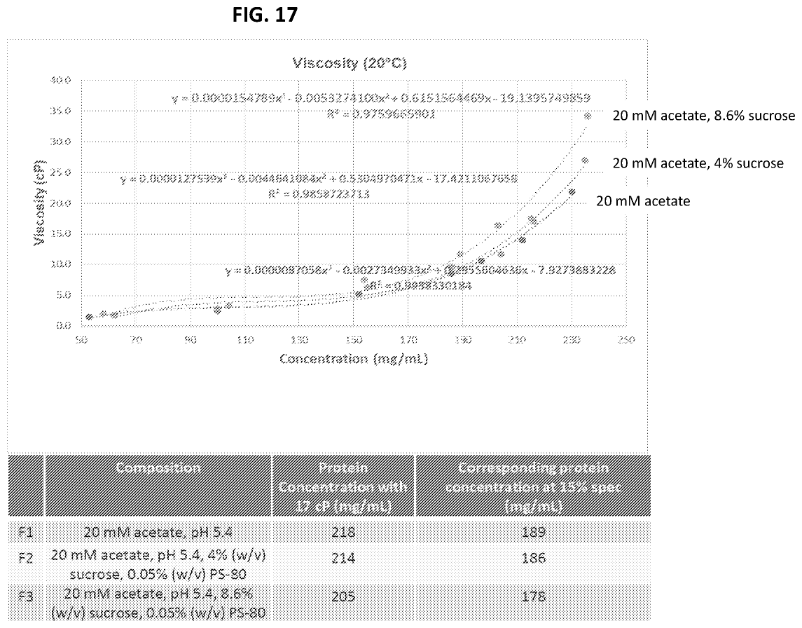

The viscosity of the compositions summarized in Table 2 were assessed for viscosity over various concentrations of fusion protein at 20° C. as shown in FIG. 17.

TABLE 2
Viscosity of Compositions

| | | Protein | Corresponding |
|---|---|---|---|
| | | Concentra- | protein |
| | | tion with | concentration |
| | | 17 cP | at 15% spec |
| | Composition | (mg/mL) | (mg/mL) |
| F1 | 20 mM acetate, pH 5.4 | 218 | 189 |
| F2 | 20 mM acetate, pH 5.4, 4% (w/v) | 214 | 186 |
| | sucrose, 0.05% (w/v) PS-80 | | |
| F3 | 20 mM acetate, pH 5.4, 8.6% (w/v) | 205 | 178 |
| | sucrose, 0.05% (w/v) PS-80 | | |

Based on the stability data, major degradation pathways were formation of HMW species at accelerated (25° C.) conditions and formation of HMW species and increased turbidity at stressed (37° C.) conditions. No significant change on charge variants was observed in these studies. A formulation concentration of 190 mg/mL was selected based on viscosity. Based on these studies, the preferred formulations were determined to be 190 mg/mL in 20 mM sodium acetate, 4% (w/v) sucrose, 0.05% (w/v) PS80, pH 5.4 as well as 190 mg/mL in 20 mM sodium acetate, 165 mM proline, 0.05% (w/v) PS80, pH 5.4. The properties of each of these formulations are summarized in Table 3.

TABLE 3
Prediction profile for preferred formulations:

| | Phase I | | |
|---|---|---|---|
| Components | formulation | Preferred | Second |
| HMW %/month@ 25° C. | 0.3 | 0.2 (0.1-0.2) | 0.2 (0.1-0.3) |
| HMW %/month@ 37° C. | 4.8 | 5.0 (4.2-5.8) | 4.6 (3.7-5.5) |
| Turbidity/month@ 37° C. | 1.3 | 3.0 (1.4-4.5) | 4.0 (2.3-5.8) |
| Viscosity @20° C. | 6.5 | 10.0 | 11.0 |

### ADDITIONAL EMBODIMENTS

All references cited in this specification, including, database-accessioned information (e.g., in GENBANK, UNIPROT, PUBMED), are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only.

What is claimed is:

1. A pharmaceutical composition comprising a fusion protein, detergent, and a buffering agent, wherein the fusion protein is at a concentration of at least 120 mg/mL.

2. The pharmaceutical composition of claim 2, wherein the fusion protein comprises complementarity-determining regions (CDRs) having amino acid sequences of SEQ ID NOS: 2-7.

3. The pharmaceutical composition of claim 2, wherein the fusion protein comprises a first portion including an amino acid sequence having at least 95% sequence identity to SEQ ID NO: 55 and a second portion including an amino acid sequence having at least 95% sequence identity SEQ ID NO: 56.

4. The pharmaceutical composition of claim 2 or 3, wherein the fusion protein consists of the amino acid sequence of SEQ ID NO: 1, or a modification thereof.

5. The pharmaceutical composition of claim 4, wherein the modification comprises conversion of the N-terminal glutamine of the sequence of SEQ ID NO: 1 to pyro-glutamate.

6. The pharmaceutical composition of any one of claims 1-5, wherein the concentration of the fusion protein is between 120 mg/mL and 250 mg/mL.

7. The pharmaceutical composition of any one of claims 1-5, wherein the fusion protein is at a concentration of at least 150 mg/mL.

8. The pharmaceutical composition of any one of claims 1-5, wherein the concentration of the fusion protein is between 150 mg/mL and 200 mg/mL.

9. The pharmaceutical composition of claim 8, wherein the concentration of the fusion protein is between 170 mg/mL and 200 mg/mL.

10. The pharmaceutical composition of claim 9, wherein the concentration of the fusion protein is about 190 mg/mL.

11. The pharmaceutical composition of claim 8, wherein the concentration of the fusion protein is about 150 mg/mL.

12. The pharmaceutical composition of any one of claims 1-11, wherein the detergent is a polysorbate.

13. The pharmaceutical composition of claim 12, wherein the polysorbate is polysorbate 80 (PS80).

14. The pharmaceutical composition of claim 12 or 13, wherein the concentration of the polysorbate is between 0.001% and 1% (w/v).

15. The pharmaceutical composition of claim 14, wherein the concentration of the polysorbate is between 0.05% (w/v) and 0.5% (w/v).

16. The pharmaceutical composition of claim 15, wherein the concentration of the polysorbate is between 0.1% (w/v) and 0.2% (w/v).

17. The pharmaceutical composition of claim 16, wherein the concentration of the polysorbate is about 0.15% (w/v).

18. The pharmaceutical composition of any one of claims 1-17, wherein the buffering agent is an acetate, a succinate, a histidine, a phosphate, or a combination thereof.

19. The pharmaceutical composition of claim 18, wherein the buffering agent is an acetate.

20. The pharmaceutical composition of claim 19, wherein the acetate is sodium acetate.

21. The pharmaceutical composition of claim 20, wherein the concentration of the sodium acetate is between about 10 mM and 150 mM.

22. The pharmaceutical composition of claim 21, wherein the concentration of the sodium acetate is between about 15 mM and 100 mM.

23. The pharmaceutical composition of claim 22, wherein the concentration of the sodium acetate is about 50 mM.

24. The pharmaceutical composition of claim 21, wherein the concentration of the sodium acetate is about 20 mM.

25. The pharmaceutical composition of any one of claims 1-24, further comprising an amino acid.

26. The pharmaceutical composition of claim 25, wherein the amino acid is arginine, proline, or glycine.

27. The pharmaceutical composition of claim 26, wherein the concentration of the amino acid is between about 100 mM and 200 mM.

28. The pharmaceutical composition of claim 27, wherein the concentration of the amino acid is about 165 mM.

29. The pharmaceutical composition of any one of claims 1-28, further comprising a tonicity agent.

30. The pharmaceutical composition of claim 29, wherein the tonicity agent is a sugar, an amino acid, or a salt.

31. The pharmaceutical composition of claim 30, wherein the salt is NaCl.

32. The pharmaceutical composition of claim 30, wherein the sugar is sucrose, glucose, glycerol, or trehalose.

33. The pharmaceutical composition of claim 32, wherein the sugar is sucrose.

34. The pharmaceutical composition of claim 32, wherein the sucrose is present at a concentration between about 1% (w/v) and about 15% (w/v).

35. The pharmaceutical composition of claim 34, wherein the sucrose is present at a concentration between 2% (w/v) and 10% (w/v).

36. The pharmaceutical composition of claim 34, wherein the sucrose is present at a concentration between 4% (w/v) and 9% (w/v).

37. The pharmaceutical composition of claim 36, wherein the sucrose is present at a concentration between 8% (w/v) and 9% (w/v).

38. The pharmaceutical composition of claim 37, wherein the sucrose is present at a concentration of about 8.6% (w/v).

39. The pharmaceutical composition of claim 34, wherein the sucrose is present at a concentration of about 4% (w/v).

40. The pharmaceutical composition of any one of claims 1-39, wherein the pharmaceutical composition has a pH of between about pH 3 and pH 8.

41. The pharmaceutical composition of claim 40, wherein the pharmaceutical composition has a pH of between pH 4 and pH 7.

42. The pharmaceutical composition of claim 41, wherein the pharmaceutical composition has a pH of about pH 5.4.

43. The pharmaceutical composition of any one of claims 1-42, wherein the pharmaceutical composition has a viscosity of between 6 cP to 35 cP at 20° C.

44. The pharmaceutical composition of any one of claims 1-43, wherein the pharmaceutical composition has a viscosity of <17 cP at 20° C.

45. The pharmaceutical composition of claim 44, wherein the pharmaceutical composition has a viscosity of <12 cP at 20° C.

46. The pharmaceutical composition of any one of claims 1-45, wherein the pharmaceutical composition has a percentage of higher molecular weight compounds per month at 25° C. over one month of between 0.05% (w/v) and 0.5% (w/v).

47. The pharmaceutical composition of claim 46, wherein the pharmaceutical composition has a percentage of higher molecular weight compounds per month at 25° C. over one month of about 0.2% (w/v).

48. The pharmaceutical composition of any one of claims 1-47, wherein the pharmaceutical composition has a percentage of higher molecular weight compounds per month at 37° C. over one month of between about 1% (w/v) and 10% (w/v).

49. The pharmaceutical composition of claim 48, wherein the pharmaceutical composition has a percentage of higher molecular weight compounds per month at 37° C. over one month of about 4.6% or about 5% (w/v).

50. The pharmaceutical composition of any one of claims 1-49, wherein the pharmaceutical composition has a turbidity of between about 1 and 10 per month at 37° C., wherein turbidity is measured as the optical density at 400 nm.

51. The pharmaceutical composition of claim 50, wherein the pharmaceutical composition has a turbidity of about 3 or about 4 per month at 37° C.

52. The pharmaceutical composition of any one of claims 1-51, wherein the pharmaceutical composition has an osmolality of between 100 Osm/kg H₂O and 500 Osm/kg H₂O.

53. The pharmaceutical composition of claim 52, wherein the pharmaceutical composition has an osmolality of about 245 Osm/kg H₂O.

54. A pharmaceutical composition comprising a fusion protein, wherein the fusion protein comprises CDRs having amino acid sequences of SEQ ID NOS: 2-7, a detergent, sucrose, and sodium acetate, wherein the fusion protein is at a concentration between 120 mg/mL and 200 mg/mL, wherein the sodium acetate is at a concentration of between 25 mM and 75 mM, wherein the sucrose has a concentration of between 2% (w/v) and 15% (w/v), wherein the detergent is at a concentration between 0.01% (w/v) and 0.2% (w/v), and wherein the pharmaceutical composition has a pH of between pH 4 and pH 7.

55. The pharmaceutical composition of claim 54, wherein the concentration of fusion protein is about 150 mg/mL, wherein the sodium acetate concentration is about 50 mM, wherein the sucrose concentration is about 8.6% (w/v), wherein the detergent is PS-80 having a concentration of about 0.05% (w/v), and wherein the pharmaceutical composition has a pH of about 5.4.

56. The pharmaceutical composition of claim 54, wherein the concentration of fusion protein is about 150 mg/mL, wherein the sodium acetate concentration is about 50 mM, wherein the sucrose concentration is about 8.6% (w/v), wherein the detergent is PS-80 having a concentration of about 0.15% (w/v), and wherein the pharmaceutical composition has a pH of about 5.4.

57. A pharmaceutical composition comprising a fusion protein, wherein the fusion protein comprises CDRs having an amino acid sequences of SEQ ID NOS: 2-7, a detergent, and sodium acetate, wherein the PS80 is at a concentration between 0.01% (w/v) and 0.1% (w/v), wherein the sucrose is at a concentration between 1% (w/v) and 10% (w/v), wherein the sodium acetate is at a concentration between 10 mM and 50 mM, and wherein the pharmaceutical composition has a pH of between pH 4 and pH 7, and wherein the fusion protein is at a concentration between 150 mg/mL and 200 mg/mL.

58. The pharmaceutical composition of claim 57, wherein the fusion protein is at a concentration of about 190 mg/mL, wherein the PS80 is at a concentration of about 0.05% (w/v) or about 0.1% (w/v), wherein the sucrose is at a concentration is 4% (w/v), wherein the sodium acetate is at a concentration of about 20 mM, and wherein the pharmaceutical composition has a pH of about pH 5.4.

59. A pharmaceutical composition comprising a fusion protein, wherein the fusion protein comprises CDRs having an amino acid sequences of SEQ ID NOS: 2-7, detergent, sodium acetate, and proline, wherein the detergent is at a concentration between 0.01% (w/v) and 0.1% (w/v), wherein the sodium acetate is at a concentration between 10 mM and 50 mM, wherein the proline is at a concentration between 100 mM and 200 mM, and wherein the pharmaceutical composition has a pH of between pH 4 and pH 7 and, wherein the fusion protein is at a concentration between 150 mg/mL and 200 mg/mL.

60. The pharmaceutical composition of claim 59, wherein the fusion protein is at a concentration of about 190 mg/mL, wherein the detergent is at a concentration of about 0.05% (w/v) or about 0.1% (w/v), wherein the sodium acetate is at a concentration of about 20 mM, wherein the proline is at a concentration of about 165 mM, and wherein the pharmaceutical composition has a pH of about pH 5.4.

61. The pharmaceutical composition of any one of claims 54-60, wherein the fusion protein comprises a first portion including an amino acid sequence having at least 95% sequence identity to SEQ ID NO: 55 and a second portion including an amino acid sequence having at least 95% sequence identity SEQ ID NO: 56.

62. The pharmaceutical composition of any one of claims 54-61, wherein the fusion protein consists of the amino acid sequence of SEQ ID NO: 1, or a modification thereof.

63. The method of claim 62, wherein the modification comprises conversion of the N-terminal glutamine of the sequence of SEQ ID NO: 1 to pyro-glutamate.

64. The pharmaceutical composition of any one of claims 1-63, wherein the pharmaceutical composition is formulated as a drug product.

65. A method of treating or prevention a disease or condition in a subject, the method comprising administering the pharmaceutical composition of claim 64 to a subject in need thereof.

66. The method of claim 65, wherein the disease is sickle cell disease.

67. The method of claim 65 or 66, wherein the pharmaceutical composition is administered subcutaneously.

68. The method of claim 67, wherein the pharmaceutical composition is administered via a pre-filled syringe, autoinjector, or on-body device.

69. The method of any one of claims 65-68, wherein the subject is human.

70. A method of developing a high concentration formulation of a fusion protein for subcutaneous administration, the method comprising:
i) performing an excipient screening, wherein the excipient screening comprises measuring stability and viscosity of a solution comprising the fusion protein and one or more excipients over a period of time;
ii) following step (i), measuring viscosity, turbidity, and formation rate of high molecular weight compounds in the solution of step (i) as a result of changing pH, acetate concentration, and sucrose concentration;
iii) following step (ii), measuring viscosity, turbidity, and formation rate of high molecular weight compounds in the solution of step (ii) as a result of changing pH, sucrose concentration, excipient, and concentration of the fusion protein; and
iv) following step (iii), performing a viscosity assessment, wherein the viscosity of the formulation is measured at a range of temperatures and a range of fusion protein concentrations.

71. The method of claim 70, wherein the fusion protein comprises 6 CDRs having amino acid sequences of SEQ ID NOS: 2-7.

72. The method of claim 70 or 71, wherein the fusion protein comprises a first portion including an amino acid sequence having at least 95% sequence identity to SEQ ID NO: 55 and a second portion including an amino acid sequence having at least 95% sequence identity SEQ ID NO: 56.

73. The method of any one of claims 70-72, wherein the fusion protein consists of the amino acid sequence of SEQ ID NO: 1, or a modification thereof.

74. The method of claim 73, wherein the modification comprises conversion of the N-terminal glutamine of the sequence of SEQ ID NO: 1 to pyro-glutamate.

75. The method of any one of claims 70-74, wherein the high concentration formulation has a fusion protein concentration of at least 120 mg/mL.

76. The method of any one of claims 70-75, wherein the high concentration formulation has a fusion protein concentration of between 120 mg/mL and 250 mg/mL.

77. The method of any one of claims 70-75, wherein the high concentration formulation has a fusion protein concentration of at least 150 mg/mL.

78. The method of any one of claims 70-77, wherein the solution of step (i) has a fusion protein concentration of about 200 mg/mL.

79. The method of any one of claims 70-78, wherein the measuring of step ii and step iii is performed weekly.

80. The method of claim 79, wherein the measuring is performed for at least 4 weeks.

81. The method any one of claims 70-80, wherein the one or more excipients is selected from succinate, arginine, acetate, histidine, phosphate, or a combination thereof.

82. The method of claim 81, wherein the one or more excipients is arginine.

83. The method of claim 82, wherein the arginine is arginine hydrochloride.

84. The method of claim 82, wherein the arginine is arginine acetate.

85. The method of claim 82 wherein the arginine is arginine succinate.

86. The method of claim 81, wherein the one or more excipients is succinate.

87. The method of claim 81, wherein the one or more excipients is succinate and arginine hydrochloride.

88. The method of claim 81, wherein the one or more excipients is acetate.

89. The method of claim 81, wherein the one or more excipients is acetate and arginine hydrochloride.

90. The method of claim 81, wherein the one or more excipients is acetate and arginine acetate.

91. The method of any one of claims 70-90, wherein the solution of step (i) further comprises sucrose.

92. The method of claim 91, wherein the sucrose is present at a concentration between 8% (w/v) and 9% (w/v).

93. The method of claim 92, wherein the sucrose is present at a concentration of about 8.6% (w/v).

94. The method of claim 91, wherein the sucrose has a concentration of between 1% (w/v) and 10% (w/v).

95. The method of claim 94, wherein the sucrose has a concentration of about 4% (w/v).

96. The method of any one of claims 70-95, wherein the solution of step (ii) has a pH of between 4 and 7.

97. The method of claim 96, wherein the solution of step (ii) has a pH of between 4.5 and 6.0.

98. The method of any one of claims 70-97, wherein the solution of step (ii) has a concentration of acetate of between 10 mM and 200 mM.

99. The method of claim 81, wherein the solution of step (ii) has a concentration of acetate of between 15 mM and 150 mM.

100. The method of any one of claims 70-99, wherein the solution of step (ii) has a concentration of sucrose of between 1% (w/v) and 20% (w/v).

101. The method of claim 100, wherein the solution of step (ii) has a concentration of sucrose of between 2% (w/v) and 10% (w/v).

102. The method of any one of claims 70-101, wherein the solution of step (ii) has a fusion protein concentration of about 150 mg/mL.

103. The method of any one of claims 70-102, wherein the solution of step (iii) has a pH of between 5 and 6.

104. The method of any one of claims 70-103, wherein the solution of step (iii) has a concentration of sucrose of between 0% (w/v) and 10% (w/v).

105. The method of claim 104, wherein the solution of step (iii) has a concentration of sucrose of between 8% (w/v) and 9% (w/v).

106. The method of any one of claims 70-105, wherein the solution of step (iii) has a fusion protein concentration of between about 100 mg/mL and 300 mg/mL.

107. The method of claim 106, wherein the solution of step (ii) has a fusion protein concentration of between about 150 mg/mL and 230 mg/mL.

108. The method of any one of claims 70-107, wherein the formulation has a viscosity of between 6 cP and 35 at 20° C.

109. The method of any one of claims 70-108, wherein the formulation has a viscosity of between 5 cP and 15 at 20° C.

110. The method of claim 109, wherein the formulation has a viscosity about 10 cP or 11 cP at 20° C.

111. The method of any one of claims 70-110, wherein the formulation has a percentage of higher molecular weight compounds per month at 25° C. over one month of between 0.05% (w/v) and 0.5% (w/v).

112. The method of claim 111, wherein the formulation has a percentage of higher molecular weight compounds per month at 25° C. over one month of about 0.2% (w/v).

113. The method of any one of claims 70-112, wherein the formulation has a percentage of higher molecular weight compounds per month at 37° C. over one month of between about 1% (w/v) and 10% (w/v).

114. The method of claim 113, wherein the formulation has a percentage of higher molecular weight compounds per month at 37° C. over one month of about 4% (w/v) or of about 5% (w/v).

115. The method of any one of claims 70-114, wherein the formulation has a turbidity of between about 1 and 10 per month at 37° C., wherein turbidity is measured as the optical density at 400 nm.

116. The method of claim 115, wherein the formulation has a turbidity of about 3 or 4 per month at 37° C.

117. A bispecific construct that binds properdin and human serum albumin, the bispecific construct comprising the amino acid sequence of SEQ ID NO:1, or a modification thereof.

118. The bispecific construct of claim 117, wherein the modification comprises conversion of the N-terminal glutamine of the amino acid sequence of SEQ ID NO:1 to pyro-glutamate.
